(19) United States
(12) Patent Application Publication
(10) Pub. No.: US 2024/0377698 A1
(43) Pub. Date: Nov. 14, 2024

(54) ELECTRO-OPTICAL MECHANICALLY FLEXIBLE NEURAL PROBES

(71) Applicants: The Regents of the University of California, Oakland, CA (US); The Salk Institute for Biological Studies, La Jolla, CA (US)

(72) Inventors: Donald Sirbuly, Carlsbad, CA (US); Spencer Ward, La Jolla, CA (US); Conor Riley, San Francisco, CA (US); Sadik Esener, Portland, OR (US); Axel Nimmerjahn, La Jolla, CA (US)

(21) Appl. No.: 18/692,757

(22) PCT Filed: Sep. 16, 2022
(86) PCT No.: PCT/US22/76614

§ 371 Date: Mar. 15, 2024

Related U.S. Application Data
(60) Provisional application No. 63/245,194, filed on Sep. 16, 2021.

(51) Int. Cl. G02F 1/29; A61N 5/06

(52) U.S. Cl. CPC G02F 1/291; A61N 5/0601; A61N 2005/0612; A61N 2005/0626; G02F 2201/02

(57) ABSTRACT

Electro-optical microprobes and methods for forming and using the electro-optical microprobes are disclosed. In one aspect, an electro-optical microprobe includes an optical waveguide including first and second ends and a side surface between the first and the second ends, a first layer including a first electrically conductive material disposed over the side surface of the optical waveguide, a second layer including an electrically conductive polymer disposed on a portion of the first layer proximate to the first end of the optical waveguide, and an isolation layer including an electrically insulative material disposed the second layer and a remaining portion of the first layer that is not covered by the second layer.

### CROSS-REFERENCE TO RELATED APPLICATION

This patent document claims priority to and benefits of U.S. Provisional Appl. No. 63/245,194, entitled “ELECTRO-OPTICAL MECHANICALLY FLEXIBLE NEURAL PROBES” and filed on Sep. 16, 2021. The entire contents of the before-mentioned patent application are incorporated by reference as part of the disclosure of this document.

### STATEMENT REGARDING FEDERALLY SPONSORED RESEARCH OR DEVELOPMENT

This invention was made with government support under HR0011-16-2-0027 awarded by Defense Advanced Research Projects Agency (DARPA). The government has certain rights in the invention.

### TECHNICAL FIELD

This patent document relates to microprobes for measuring neurons.

### BACKGROUND

Microelectrodes are the gold standard for measuring the activity of individual neurons at high temporal resolution in any nervous system region and central to defining the role of neural circuits in controlling behavior. Existing microelectrode technologies have allowed tracking of distributed neural activity with millisecond precision. However, their large footprint and rigidity lead to tissue damage and inflammation that hamper long-term recordings.

### SUMMARY

Disclosed are multi-modal coaxial microprobes with a minimally invasive footprint that enables efficient electrical and optical interrogation of neural networks.

In an implementation of the disclosed technology, an electro-optical microprobe includes an optical waveguide including first and second ends and a side surface between the first and the second ends, a first layer including a first electrically conductive material disposed over the side surface of the optical waveguide, a second layer including an electrically conductive polymer disposed on a portion of the first layer proximate to the first end of the optical waveguide, and an isolation layer including an electrically insulative material disposed the second layer and a remaining portion of the first layer that is not covered by the second layer.

In another implementation of the disclosed technology, a method of manufacturing an electro-optical coaxial microprobe includes providing an optical waveguide including first and second ends and a side surface between the first and the second ends, forming a first layer including a first electrically conductive material over the side surface of the optical fiber, forming a second layer including an electrically conductive polymer on a portion of the first layer proximate to the first end of the optical waveguide, and forming an isolation layer including an electrically insulative polymer on the second layer and a remaining portion of the first layer that is not covered by the second layer.

In another implementation of the disclosed technology, a method of using the electro-optical coaxial microprobe includes interfacing the microprobe with one or more neural networks, providing optogenetic stimulation, and conducting electrical measurements associated with a neural activity of the one or more neural networks, wherein the electro-optical coaxial microprobe has a sufficiently small length and diameter to produce negligible inflammatory response.

Those and other features are described in greater detail in the drawings, the description and the claims.

### BRIEF DESCRIPTION OF THE DRAWINGS

FIGS. 1A-1H show examples of implantable electro-optical mechanically flexible (EO-Flex) probes along with optical and electrical characterization.

FIGS. 2A-2E show extracellular neural recordings in the cortex of live mice using the EO-Flex probes.

FIGS. 3A-3Q show measurement of whisker stimulation-induced sensory activity in the barrel cortex of awake head-restrained mice on a spherical treadmill using the EO-Flex probes.

FIGS. 4A-4H show concomitant optical stimulation and electrical recording with the EO-Flex probes in live mice.

FIGS. 5A-5N show EO-Flex probes evoke minimal tissue responses compared to multimode fibers commonly used in optogenetic experiments.

FIGS. 6A-6G show finite element modelling of electromagnetic (EM) modes to investigate the theoretical optical coupling between the single-mode fiber (SMF) and microfiber.

FIGS. 7A-7D show electron micrographs of four microfiber EO-Flex probes with different PEDOT:PSS deposition conditions. FIG. 7E shows a table of the microfiber core diameters along with the different thicknesses for each layer deposited on the probes.

FIGS. 8A-8C show EO-Flex probes fabricated with a single crystalline tin dioxide (SnO2) nanofiber waveguide as the optical core.

FIG. 9 shows EO-Flex recordings across cortical layers of an anesthetized mouse.

Insertion depths calculated from stereotactic coordinates are displayed with corresponding recordings.

FIGS. 10A-10D show electrical stimulation evoked whisker deflection with EO-Flex probes.

FIG. 11 shows EO-Flex testing in layer 2/3 of a live mouse as a function of optical stimulation power.

FIGS. 12A-12D show estimated light intensity distribution and heating around the EO-Flex probe tip for continuous or pulsed 470 nm EO-Flex light of 208 μW or 1 mW.

FIG. 13 shows EO-Flex testing in layer 2/3 of a live mouse as a function of the optical pulse width.

FIG. 14 shows EO-Flex testing in layer 2/3 of a live mouse as a function of stimulation frequency.

FIG. 15 EO-Flex optical testing as a function of different optical stimulation frequencies (5, 10, 20, 30, 40, and 50 Hz) in an awake mouse without light-activated protein expression.

FIG. 16 shows EO-Flex testing in a live mouse as a function of recording depth.

FIGS. 17A-17E show in vivo calcium imaging combined with simultaneous electrical recordings in the cortex of mice confirms EO-Flex mediated optical excitation of neurons.

FIG. 18 shows chronic recordings with EO-Flex probes.

FIGS. 19A-19B show EO-Flex probes evoke minimal tissue inflammatory responses compared to multimode fibers commonly used in optogenetic experiments.

FIGS. 20A-20B show EO-Flex probes evoke minimal tissue inflammatory responses at 30 days post implant when compared to larger multimode fibers commonly used in optogenetic experiments.

FIGS. 21A-21E show EO-Flex probes evoke minimal tissue responses compared to multimode fibers commonly used in optogenetic experiments.

FIGS. 22A-22C show scaling strategies of the EO-Flex probes for both probe length and arrays.

FIG. 23 shows an example of an electro-optical microprobe based on some embodiments of the disclosed technology.

FIG. 24 shows an example method of manufacturing an electro-optical coaxial microprobe based on some embodiments of the disclosed technology.

### DETAILED DESCRIPTION

Central to advancing our understanding of neural circuits is the development of minimally invasive, multi-modal interfaces capable of simultaneously recording and modulating neural activity. Recent devices have focused on matching the mechanical compliance of tissue to reduce inflammatory responses. However, reductions in the size of multi-modal interfaces are needed to further improve biocompatibility and long-term recording capabilities. The disclosed technology can be implemented in some embodiments to provide a multi-modal coaxial microprobe design with a minimally invasive footprint (e.g., 8-12 μm diameter over millimeter lengths) that enables efficient electrical and optical interrogation of neural networks. In the brain, the probes based on some embodiments of the disclosed technology allow robust electrical measurement and optogenetic stimulation. Scalable fabrication strategies can be used with various electrical and optical materials, making the probes highly customizable to experimental requirements, including length, diameter, and mechanical properties. Given their negligible inflammatory response, the probes based on some embodiments of the disclosed technology promise to enable a new generation of readily tunable multi-modal devices for minimally invasive interfacing with neural circuits.

Microelectrode recordings are the gold standard for measuring individual neurons' activity at high temporal resolution in any nervous system region and central to defining the role of neural circuits in controlling behavior. Microelectrode arrays, such as the Utah or Michigan arrays, have allowed tracking of distributed neural activity with millisecond precision. However, their large footprint and rigidity lead to tissue damage and inflammation that hamper long-term recordings. State of the art Neuropixel and carbon fiber probes have improved on these previous devices by increasing electrode density and reducing probe dimensions and rigidity. Although these probes have advanced the field of recordings, next-generation devices should enable targeted stimulation in addition to colocalized electrical recordings. Optogenetic techniques enable high-speed modulation of cellular activity through targeted expression and activation of light-sensitive opsins. However, given the strong light scattering and high absorption properties of neural tissue optogenetic interfacing with deep neural circuits typically requires the implantation of large-diameter rigid fibers, which can make this approach more invasive than its electrical counterpart.

The ideal neural probe would combine optical and electrical modes while maintaining small cross-sectional dimensions and tunable lengths. The ability to bi-directionally interface with genetically defined neuron types and circuits is key to ultimately being able to understand how the nervous system computes and controls behavior. It is also fundamental for determining the mechanistic basis of sensorimotor disorders, defining how circuit activity is affected by injury, and how it might be restored or facilitated. Approaches to integrating optical and electrical modalities have ranged from adding fiber optics to existing Utah arrays to the Optetrode or other integrated electro-optical coaxial structures. These technologies have shown great promise for simultaneous electrical recordings and optical stimulation in vivo. However, the need to reduce the device footprint to minimize immune responses for long-term recordings is still present.

The disclosed technology can be implemented in some embodiments to provide a coaxial neural probe with a low impedance electrical channel surrounding a small central fiber optic core. In some implementations, the electro-optical mechanically flexible (EO-Flex) probes can be fabricated with diameters as small as 8 μm and lengths up to several millimeters using microfiber optic waveguide cores or even smaller diameters with nanofiber optic cores. They can be bonded directly to single-mode fibers (SMFs) to create detachable, low-loss optical interfaces that can be directly connected to standard optogenetic hardware. The EO-Flex probes' simultaneous electrical recording and optical stimulation performance can be demonstrated in the mouse brain. In some implementations, the porous metal electrical channel provides excellent recording ability even with the probe's small size. The low source-to-tip optical losses of <10 dB allow robust optogenetic stimulation in transgenic or virally transduced mice expressing opsins in target cells. Implant studies show minimal immune responses, suggesting that the fully customizable probe and future high-density arrays should enable long-term interfacing with minimal disturbance to the surrounding neural tissue.

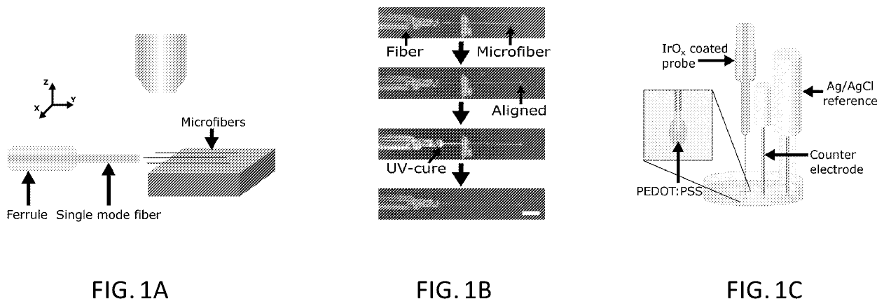

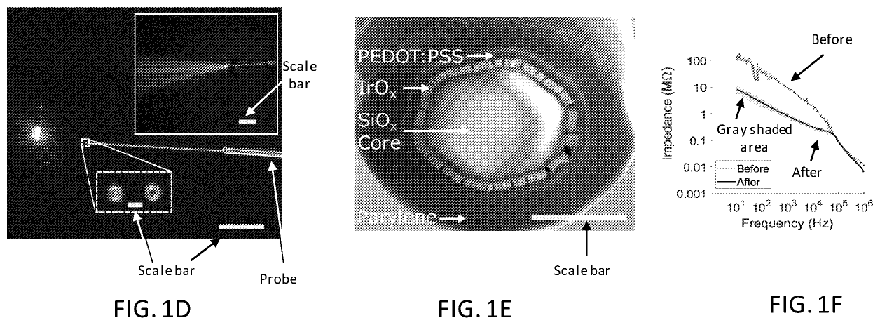

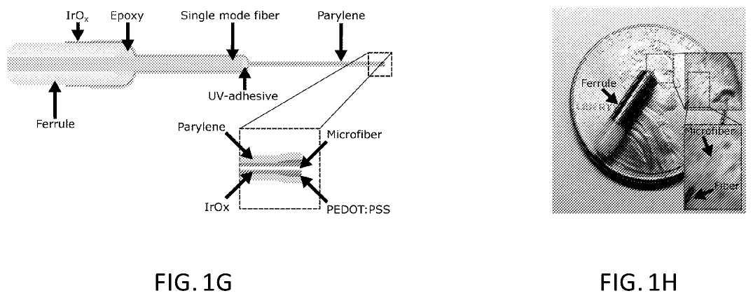

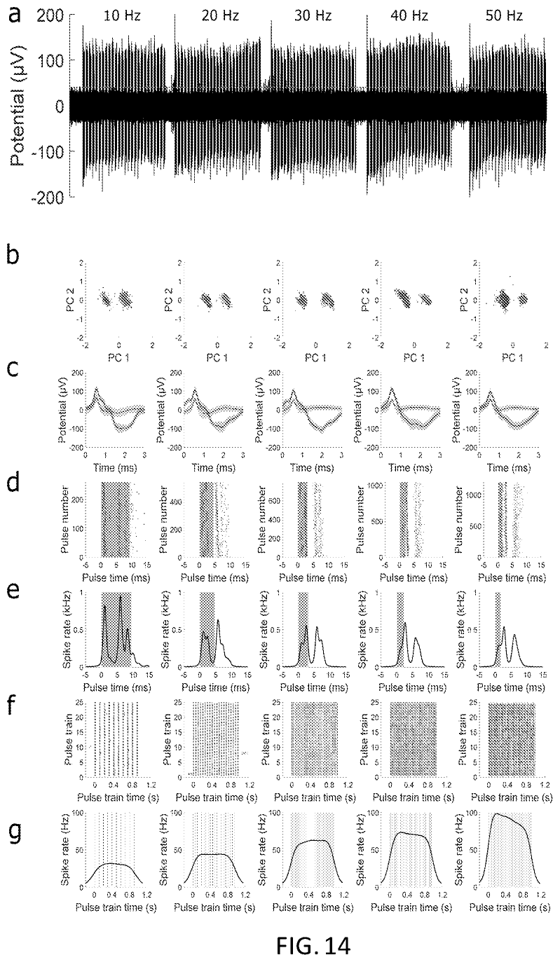

FIGS. 1A-1H show examples of implantable electro-optical mechanically flexible (EO-Flex) probes along with optical and electrical characterization. Specifically, FIG. 1A shows silica microfibers of defined length that are positioned on a silicon substrate to allow a single-mode fiber (SMF)-loaded ferrule to bond to the microfiber. FIG. 1B shows, from top to bottom, active alignment and bonding process of coupling the microfiber to the SMF (scale bar is 250 μm). FIG. 1C shows an example of the electrodeposition set-up for depositing PEDOT:PSS after the metal sputtering step (not shown). FIG. 1D shows an optical image of the light output of a probe from the side as the light reflects from a mirror, and from the cleaved end-facet (zoom-in insets) with and without laser light. The inset shows fluorescence image capturing the cone angle of the probe after submerging it in a dye solution and launching blue (442 nm) light into the probe. The scale bars are 500 μm, 12 μm, and 60 μm respectively. FIG. 1E shows a cross-sectional electron micrograph of an EO-Flex probe after milling the end showing the exposed conductive rings along with the optical core. The scale bar is 4 μm. FIG. 1F shows EIS data for milled probes with and without the PEDOT:PSS cladding. The gray shaded area is one standard deviation; n=4 probes. FIG. 1G shows a cross-sectional view of the probe showing its various cladding layers. FIG. 1H shows a completed EO-Flex probe with a zoom-in of the microfiber tip region. Scaling strategies will be discussed below with reference to FIGS. 14A-14C.

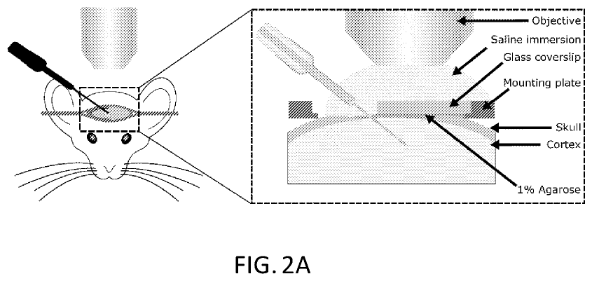

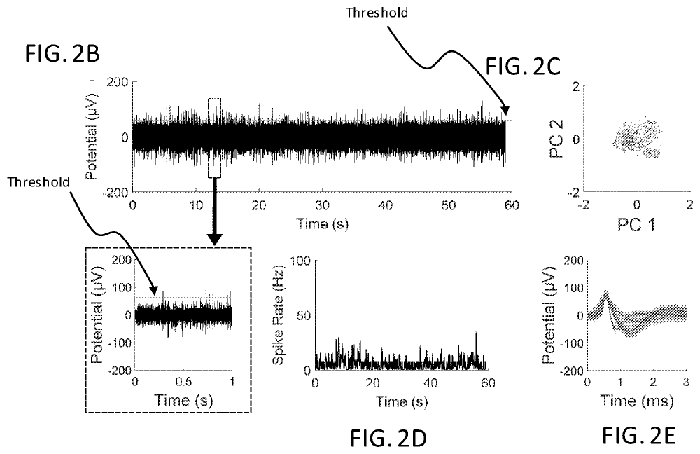

FIGS. 2A-2E show extracellular neural recordings in the cortex of live mice using the EO-Flex probes. Specifically, FIG. 2A shows the set-up used for visually guided electrical measurements. Two-photon imaging of the probe in relation to fluorescently labeled cells can be used to verify and optimize the recording position. The inset in FIG. 2A shows a zoom-in cross-sectional view of the surgical preparation for simultaneous imaging and electrical recordings. FIG. 2B shows an example EO-Flex recording showing spontaneous neural activity in cortical layer 2/3 (depth=250 μm) of an isoflurane-anesthetized mouse. The threshold defining a spike can be set to Threshold=4*median(|Recording|/0.675) based on published literature. The boxed region from the recording shows multi-unit activity. FIG. 2C shows a principle component analysis (PCA) plot of the waveform clustering using established clustering methods. FIG. 2D shows a spiking rate over the one-minute recording shown in FIG. 2B calculated using a Bayesian kernel estimation. FIG. 2E shows average waveforms (solid lines) along with one standard deviation (shaded regions) for four clusters determined by PCA from the recording in FIG. 2B.

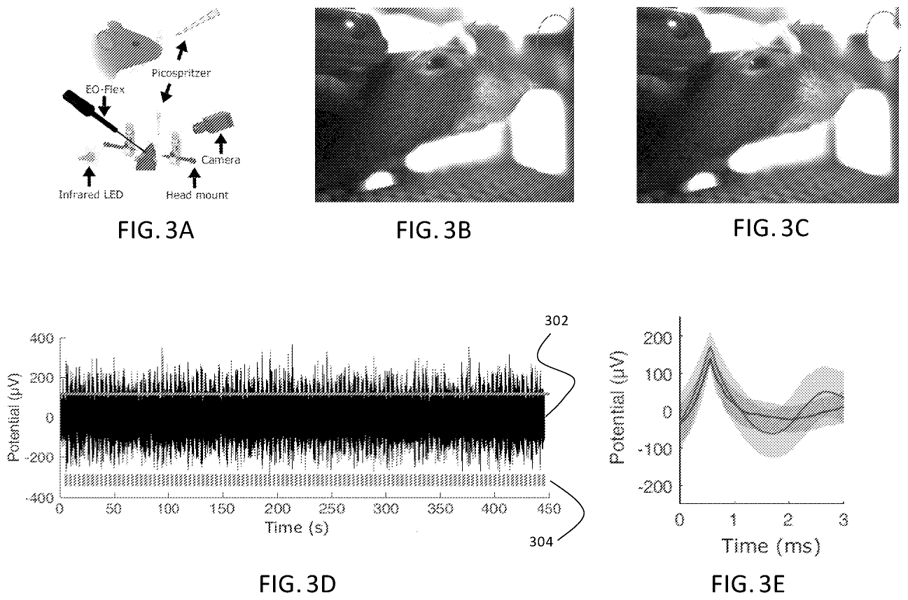

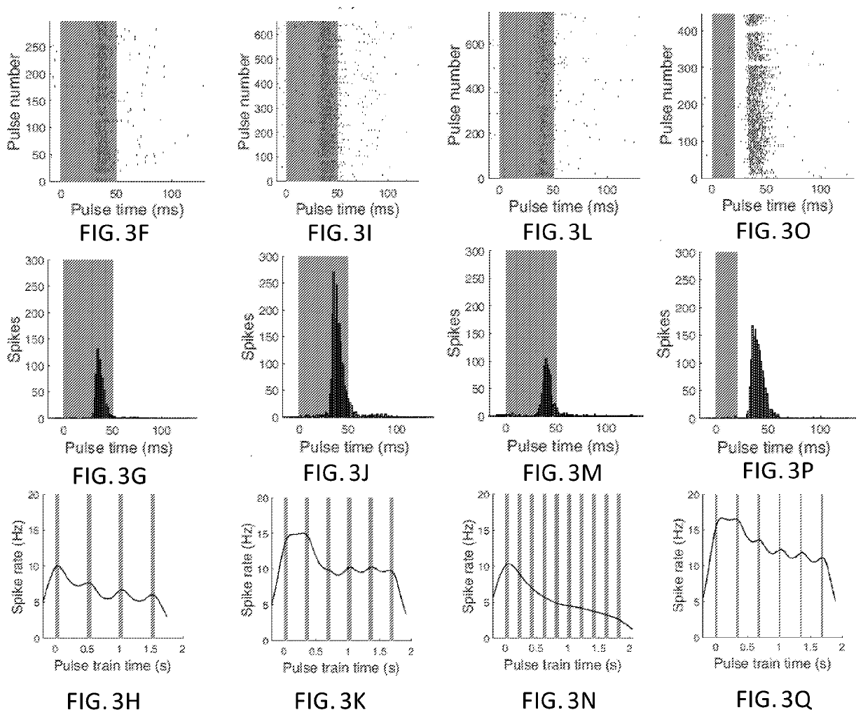

FIGS. 3A-3Q show measurement of whisker stimulation-induced sensory activity in the barrel cortex of awake head-restrained mice on a spherical treadmill using the EO-Flex probes. Specifically, FIG. 3A shows schematic of the experimental setup. Whiskers were deflected by air puff stimuli delivered through a micropipette connected to a function generator-controlled pressure system. The function generator also controlled an infrared LED for analog and video data synchronization. FIG. 3B shows the resting animal before whisker deflection. The blue arrow indicates an example whisker. The red circle indicates the infrared LED's location. FIG. 3C shows deflection of the indicated whisker during air puff delivery. FIG. 3D shows an example EO-Flex recording (302) during 3 Hz whisker stimulation (304). FIG. 3E shows corresponding spike sorted average waveforms with one standard deviation shaded. Different neural waveforms are marked with different colors. FIGS. 3F-3H: raster plot showing whisker stimulus-evoked activity for a 2 Hz stimulation frequency (50 ms pulse width) (FIG. 3F), peristimulus time histogram (FIG. 3G), and BAKS estimation of firing frequency (FIG. 3H). FIGS. 31-3K: raster plot (FIG. 3I), peristimulus time histogram (FIG. 3J), and BAKS estimation (FIG. 3K) for a 3 Hz stimulation (50 ms pulse width). FIGS. 3L-3N: raster plot (FIG. 3L), peristimulus time histogram (FIG. 3M), and BAKS estimation (FIG. 3N) for 5 Hz stimulation (50 ms pulse width). FIGS. 3O-3Q: raster plot (FIG. 3O), peristimulus time histogram (FIG. 3P), and BAKS estimation (FIG. 3Q) for 3 Hz stimulation (20 ms pulse width).

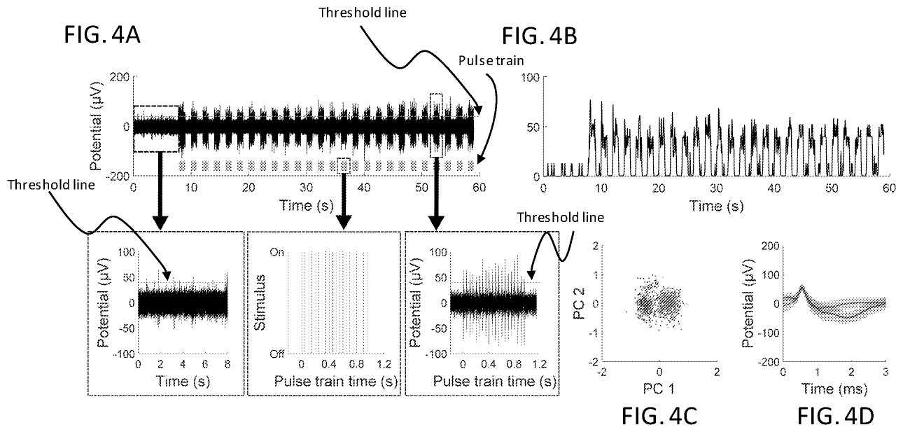

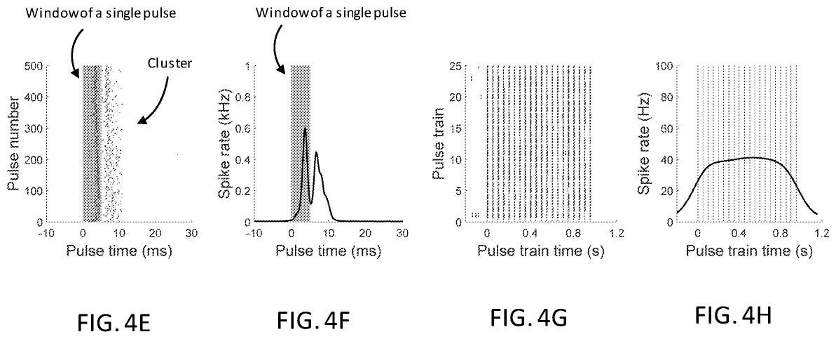

FIGS. 4A-4H show concomitant optical stimulation and electrical recording with the EO-Flex probes in live Thy1-ChR2-YFP mice. Specifically, FIG. 4A shows an example of optically evoked neural activity using a 20 Hz pulse train of 473 nm light (pulse width of 4.95 ms) at a tip power of 61 μW that is cycled on and off at 1 Hz. The threshold line can be set as defined in FIGS. 2A-2E. FIG. 4B shows a spike rate plot for the recording in FIG. 4A. FIG. 4C shows a PCA plot for the optically evoked neural activity. FIG. 4D shows average waveforms (solid line) for each cluster in FIG. 4C along with one standard deviation (shaded region). FIG. 4E shows each cluster plotted over time along with the window of a single pulse. FIG. 4F shows Bayesian kernel smoothing estimate of spiking rate for each stimulation period. FIG. 4G shows a raster plot showing occurrence of waveforms from FIG. 4D. FIG. 4H shows a calculated average spike rate over the 1 s duration of a single pulsing cycle.

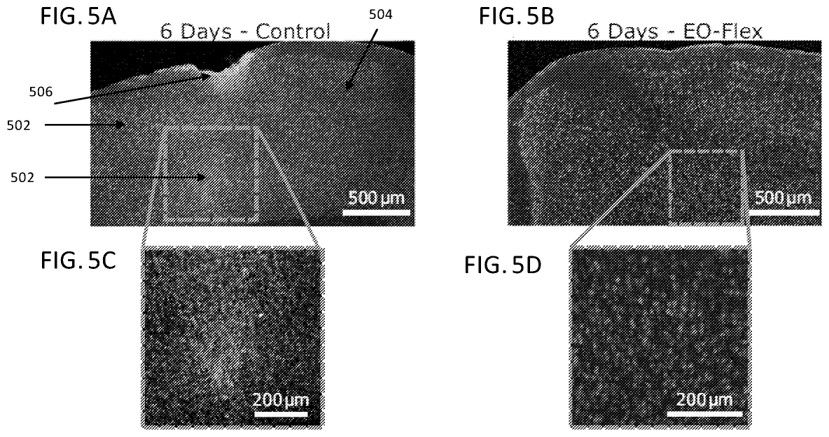

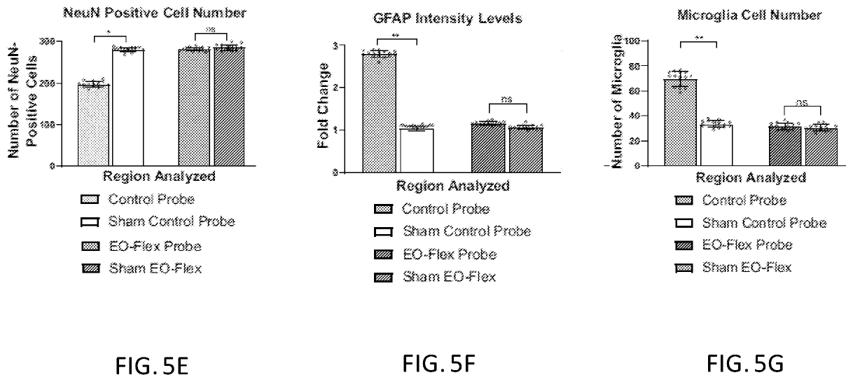

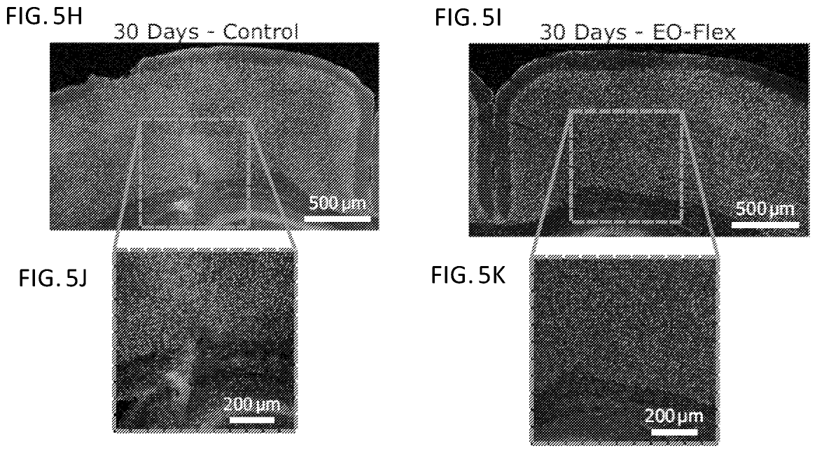

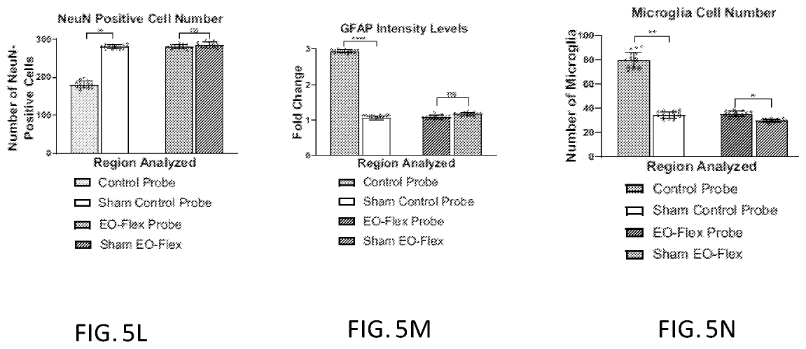

FIGS. 5A-5N show EO-Flex probes evoke minimal tissue responses compared to multimode fibers commonly used in optogenetic experiments. FIGS. 5A-5B show optical images showing 20 μm thick coronal brain sections around the multimode fiber (FIG. 5A) and EO-Flex probe (FIG. 5B) implantation sites. Both the multimode fiber (diameter, 250 μm) and EO-Flex probe (diameter, 12 μm) are advanced to an ˜1 mm depth into the cortex. The images are taken 6 days after implantation in heterozygous Cx3cr1-GFP mice with labeled microglia (502). The sections were co-stained with anti-NeuN (504) and anti-GFAP (506) antibodies to label neurons and astrocytes, respectively. FIGS. 5C-5D show higher resolution images of a zoomed-in region of (FIGS. 5A-5B) where the probe tips were located. FIGS. 5E-5G are population analysis (n=16 sections from two animals) showing the impact of the multimode fiber or EO-Flex probe implantation on neuronal cell numbers (FIG. 5E), astrocyte reactivity as measured by GFAP expression level (FIG. 5F), and microglia reactivity as measured by microglial cell number (FIG. 5G) for the 6-day implantation. Cellular responses were quantified and averaged across two 150 μm×1 mm analysis regions flankingeach insertion site. To distinguish surgery from probe-related tissue responses, an additional craniotomy of comparable size was made 0.7 mm lateral to each device implantation site. The same analysis approach was used to quantify cellular responses at this sham surgery site. Two-tailed paired t-tests determined P values. FIGS. 5H-5I show coronal brain section optical images taken 30 days after multimode fiber (FIG. 5H) and EO-Flex probe (FIG. 5I) implantation. FIGS. 5J-5K show higher resolution images of a zoomed-in region of (FIG. 5H) and (FIG. 5I) where the probe tips were located. FIGS. 5L-5N show corresponding population analysis (n=16 sections from two animals) showing neuronal cell numbers (FIG. 5L), astrocyte reactivity (FIG. 5M), and microglia reactivity (FIG. 5N) for the 30-day implantation. Two-tailed paired t-tests determined P values. The following convention was used to indicate P values: “ns” indicates P>0.05, “*” indicates 0.01<P≤0.05, “**” indicates 0.001<P≤0.01, and “****” indicates 0.0001<P≤0.001. All bar plots are presented as mean±s.e.m.

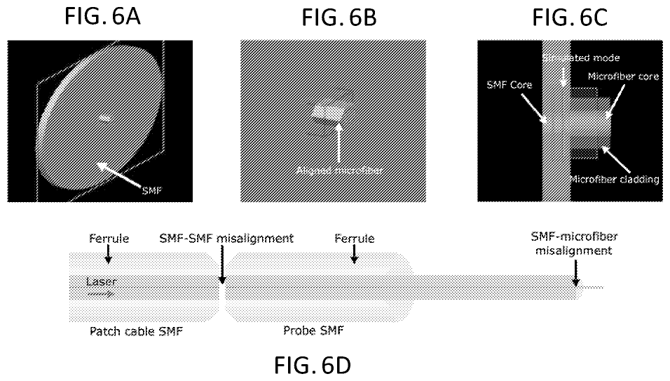

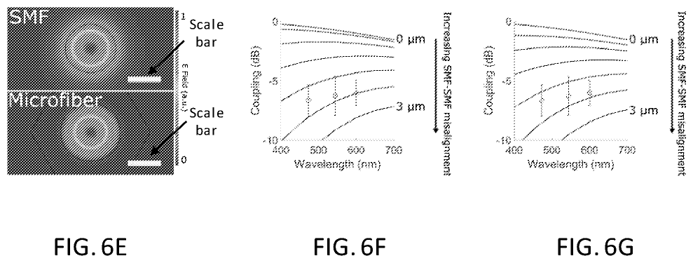

FIGS. 6A-6G show finite element modelling of electromagnetic (EM) modes (e.g., Lumerical MODE) to investigate the theoretical optical coupling between the single-mode fiber (SMF) and microfiber. FIG. 6A shows simulation geometry of an SMF fiber that has its core perfectly aligned with the core of the microfiber. FIG. 6B shows zoom-in of the SMF-microfiber interface showing a 10 nm mesh used to quantify the coupling efficiency. FIG. 6C shows a side profile of FIG. 6B with design and labels of the model. FIG. 6D shows schematic of the optical coupling into the probe from the laser source showing where the mode misalignment is simulated (SMF-SMF and SMF-microfiber). FIG. 6E shows a mode profile simulated at 473 nm for both the SMF and the microfiber. Scale bars are 2 μm. FIG. 6F shows simulated coupling efficiency assuming no misalignment between the SMF-microfiber interface and misalignment of the SMF-SMF interface from 0 to 3 μm (solid lines indicate every 0.5 μm of misalignment). Average coupling of 4 probes measured at 473 nm, 543 nm, and 600 nm are overlaid on simulated curves. FIG. 6G shows calculated coupling efficiency assuming the SMF-microfiber interface has 500 nm of misalignment and similar misalignment of the SMF-SMF interface as in FIG. 6F. The same measured data set in FIG. 6F is overlaid on the new coupling curves. Comparison of simulation and measurements shows that optical coupling losses are mostly due to SMF-SMF misalignment (measured losses fall between 2-2.5 μm of SMF-SMF misalignment).

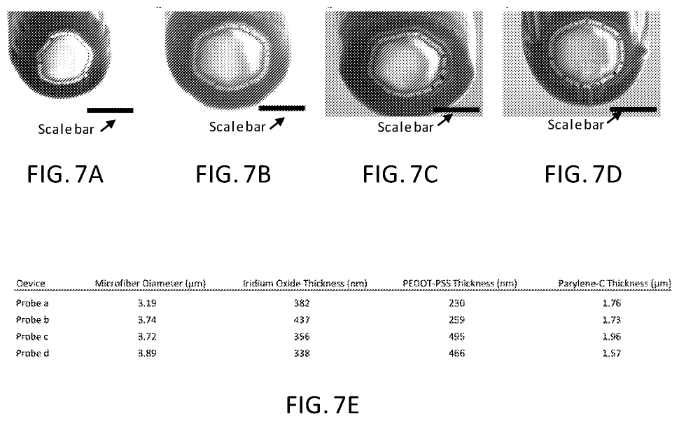

FIGS. 7A-7D show electron micrographs of four microfiber EO-Flex probes with different PEDOT:PSS deposition conditions. Data may be used to optimize the polymer thickness and quantify the dimensions of the other cladding layer. Scale bars are 5 μm (FIG. 7A) and 4 μm (FIGS. 7B-7D). FIG. 7E shows a table of the microfiber core diameters along with the different thicknesses for each layer deposited on the probes.

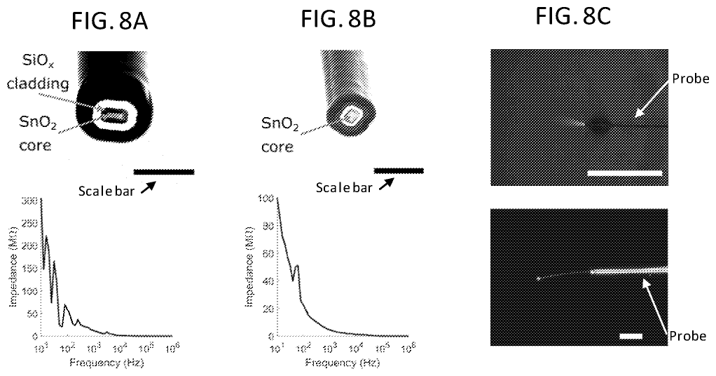

FIGS. 8A-8C show EO-Flex probes fabricated with a single crystalline tin dioxide (SnO2) nanofiber waveguide as the optical core. FIG. 8A shows at top an electron micrograph of a SnO2 EO-Flex probe fabricated without the PEDOT-PSS layer and at bottom EIS data of the probe showing an impedance of >20 MΩ at 1 kHz. Scale bar is 5 μm. FIG. 8B shows at top an electron micrograph of a SnO2 EO-Flex probe fabricated with the PEDOT-PSS layer and at bottom EIS data of the probe showing a significant reduction in the impedance down to 5 MΩ at 1 KHz. Scale bar 10 μm. FIG. 8C shows at top the optical output of a SnO2 EO-Flex probe in a fluorescent dye solution showing the exiting cone angle (scale bar is 250 μm) and at bottom an optical image of the freestanding probe showing no light scattering at the SnO2-SMF interface after cladding deposition (scale bar is 250 μm).

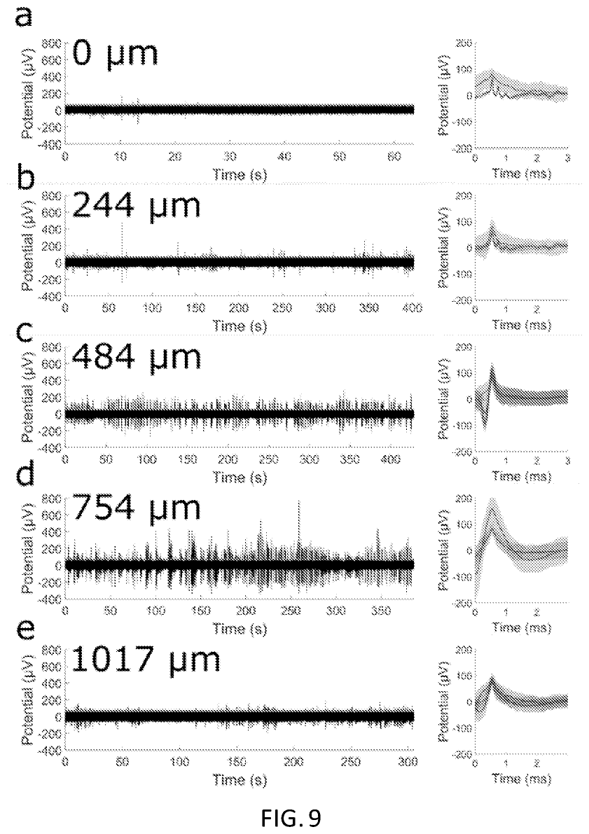

FIG. 9 shows EO-Flex recordings across cortical layers of an anesthetized mouse. Insertion depths calculated from stereotactic coordinates are displayed with corresponding recordings. In FIG. 9, (a)-(e) show example recordings showing spontaneous activity at different probe insertion depths. Corresponding spike sorted average waveforms with one standard deviation (shaded) are shown on the right. Reduced spike amplitude and activity at 1017 μm insertion depth suggests entrance into the white matter beneath the cortex.

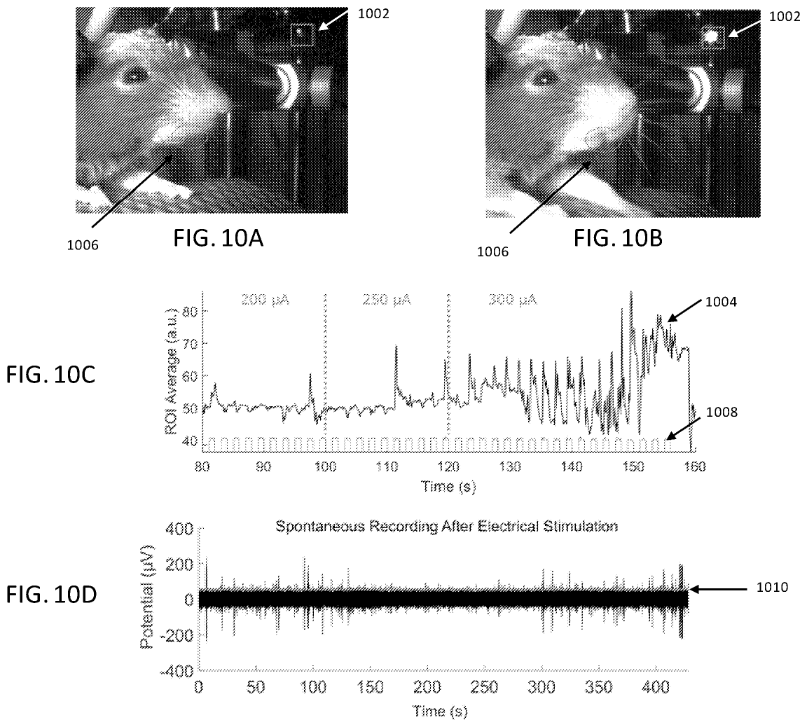

FIGS. 10A-10D show electrical stimulation evoked whisker deflection with EO-Flex probes. In this example from an awake head-restrained mouse on a spherical treadmill, the probe was implanted into the barrel cortex for 30 days with its tip inserted to a depth of 761 μm. Stimulation current was ramped up from 0 μA to 300 μA while using a stimulation frequency of 100 Hz, pulse width of 0.2 ms, and 1 Hz stimulation period. Specifically, FIG. 10A shows the resting animal before electrical stimulation. Electrical pulse train delivery was controlled by a function generator. This device also controlled an infrared LED (1002) for analog and video data synchronization. The red ROI indicates an analysis region around a group of whiskers. FIG. 10B shows whisker pad deflection during stimulus delivery. FIG. 10C shows average pixel intensity (1004) in the indicated ROI (1006) as the current is increased in 50 μA steps every 20 s. The timing of stimulus delivery is indicated at the bottom (1008). FIG. 10D shows spontaneous recording acquired after the electrical stimulation demonstrating that the EO-Flex probe remained intact. Spikes were detected when the potential crosses the threshold (horizontal straight line 1010).

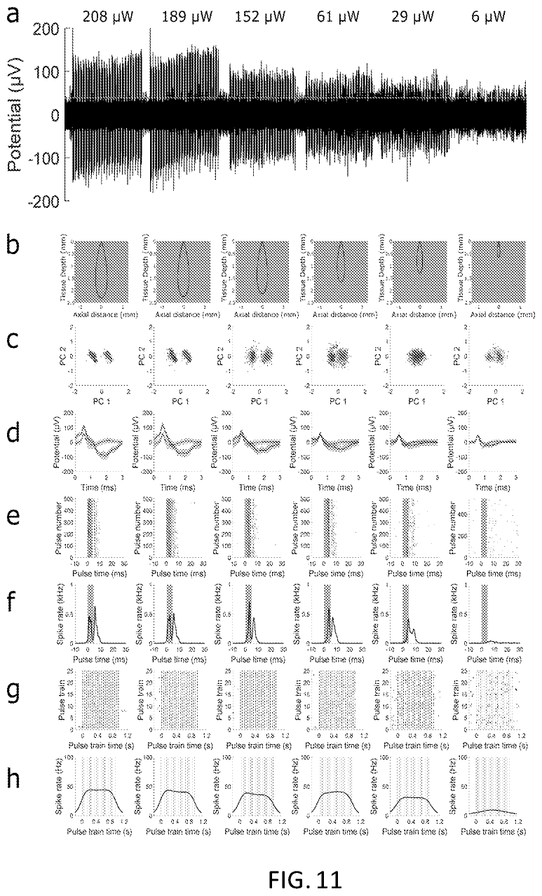

FIG. 11 shows EO-Flex testing in layer 2/3 of a live Thy1-ChR2-YFP mouse as a function of optical stimulation power. All other stimulation parameters are held constant (pulse width, 4.5 ms; stimulation frequency, 20 Hz; on/off cycling, 1 Hz). Recording depth is ˜250 μm. FIG. 11(a) shows an optically evoked neural activity using EO-Flex output powers ranging from 208 μW (20,435 mW mm-2) to 6 μW (560 mW mm-2). FIG. 11(b) shows Monte Carlo simulations of the scattering and absorption in neural tissue for each of the power values in FIG. 11(a). The solid line indicates where irradiance has fallen to 1 mW mm-2. The simulations show that a light intensity of 208 μW could propagate up to 2.4 mm from the probe tip before the irradiance dropped below 1 mW mm-2. Simulation parameters are taken from recent studies 11 which estimated scattering and absorption coefficients of 0.125 mm-1 and 7.37 mm-1, respectively. FIG. 11(c) shows the first two principal components (PCs) of respective electrical recordings plotted with the Calinski-Harabasz metric for determination of the number of clusters for mixed Gaussian fitting FIG. 11(d) shows the average waveform for each cluster (solid line) from FIG. 11(c) with the shaded region representing one standard deviation. FIG. 11(e) shows peri-stimulus plots for all optical pulses with spikes color-coordinated with the cluster from which they come. FIG. 11(f) shows Bayesian adaptive kernel smoother (BAKS) estimation for the firing rate over the time window around the optical pulses. FIG. 11(g) shows peri-stimulus plot for each optical pulse train from high to low power. FIG. 11(h) shows BAKS estimation for the firing rate over the pulse train window.

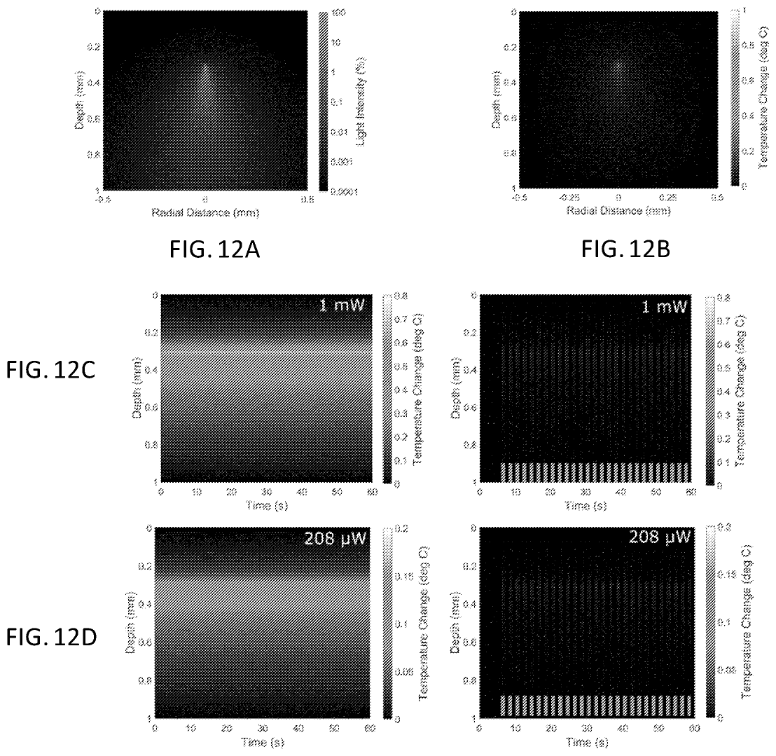

FIGS. 12A-12D show estimated light intensity distribution and heating around the EO-Flex probe tip for continuous or pulsed 470 nm EO-Flex light of 208 μW or 1 mW. For time-based simulations (FIGS. 12C-12D), the average temperature change was calculated within a cylinder of 20 μm radius around the probe with the probe tip located at a 0.3 mm depth. The results were obtained using previously validated optogenetic heating simulation software. Specifically, FIG. 12A shows light output distribution for an EO-Flex probe whose tip is located at a 0.3 mm depth within a 1×1 mm simulation volume. FIG. 12B shows heating profile around the EO-Flex probe tip pumped with continuous 470 nm light at 208 μW for 60 s (maximum temperature 0.34° C.). FIG. 12C shows average temperature around the EO-Flex probe as a function of time for continuous (left, max 0.66° C.) and 20 Hz pulsed with 1 Hz on/off cycling (right, max 0.37° C.) of 470 nm light at a power of 1 mW. FIG. 12D shows average temperature around the EO-Flex probe as a function of time for continuous (left, max 0.14° C.) and 20 Hz pulsed with 1 Hz on/off cycling (right, max 0.08° C.) of 470 nm light at a power of 208 μW.

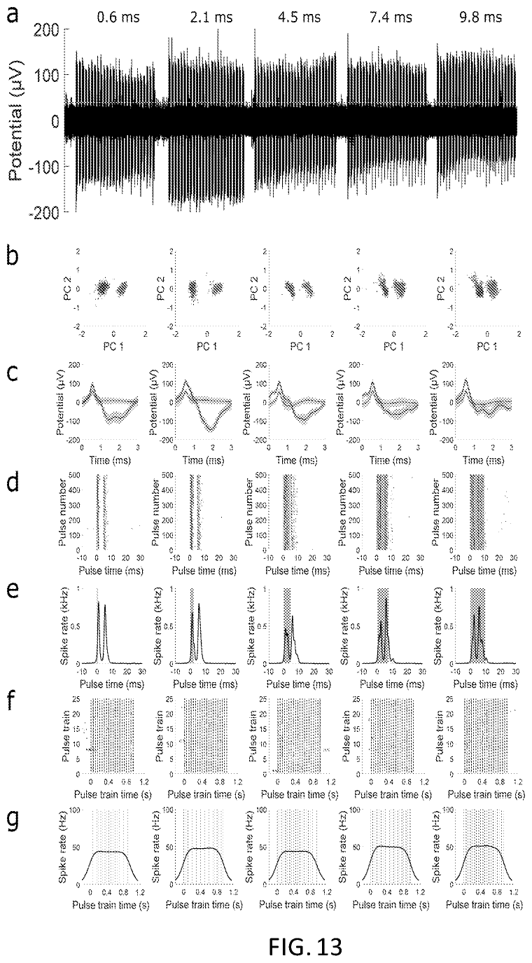

FIG. 13 shows EO-Flex testing in layer 2/3 of a live Thy1-ChR2-YFP mouse as a function of the optical pulse width. All other stimulation parameters are held constant (optical stimulation power, 208 μW; stimulation frequency, 20 Hz; on/off cycling, 1 Hz). Recording depth is ˜250 μm. FIG. 13(a) shows an optically evoked neural activity using pulse widths ranging from 0.6 ms to 9.8 ms. FIG. 13(b) shows the first two principal components (PCs) of respective electrical recordings plotted with the Calinski-Harabasz metric for determining the number of clusters for mixed Gaussian fitting. FIG. 13(c) shows an average waveform (solid line) for each cluster from FIG. 13(b) with the shaded region representing one standard deviation. FIG. 13(d) shows peri-stimulus plots for all optical pulses with spikes color-coordinated with the cluster from which they come. FIG. 13(e) shows Bayesian adaptive kernel smoother (BAKS) estimation of firing rate over the time window around the optical pulses. FIG. 13(f) shows peri-stimulus plot for each optical pulse train as a function of the optical pulse width. FIG. 13(g) shows BAKS estimation for the firing rate over the pulse train window.

FIG. 14 shows EO-Flex testing in layer 2/3 of a live Thy1-ChR2-YFP mouse as a function of stimulation frequency. All other stimulation parameters are held constant (optical stimulation power, 208 μW; duty cycle, 10%; on/off cycling, 1 Hz). Recording depth is ˜250 μm. FIG. 14(a) shows an optically evoked neural activity using stimulation frequencies ranging from 10 Hz to 50 Hz. FIG. 14(b) shows the first two principal components (PCs) of respective electrical recordings plotted with the Calinski-Harabasz metric for determining the number of clusters for mixed Gaussian fitting. FIG. 14(c) shows an average waveform (solid line) for each cluster from FIG. 14(b) with the shaded region representing one standard deviation. FIG. 14(d) shows peri-stimulus plots for all optical pulses with spikes color-coordinated with the cluster from which they come. FIG. 14(e) shows Bayesian adaptive kernel smoother (BAKS) estimation of the firing rate over the short time window around the optical pulses. FIG. 14(f) shows peri-stimulus plot for each optical pulse as a function of the stimulation frequency. FIG. 14(g) shows BAKS estimation for the firing rate over the pulse train window.

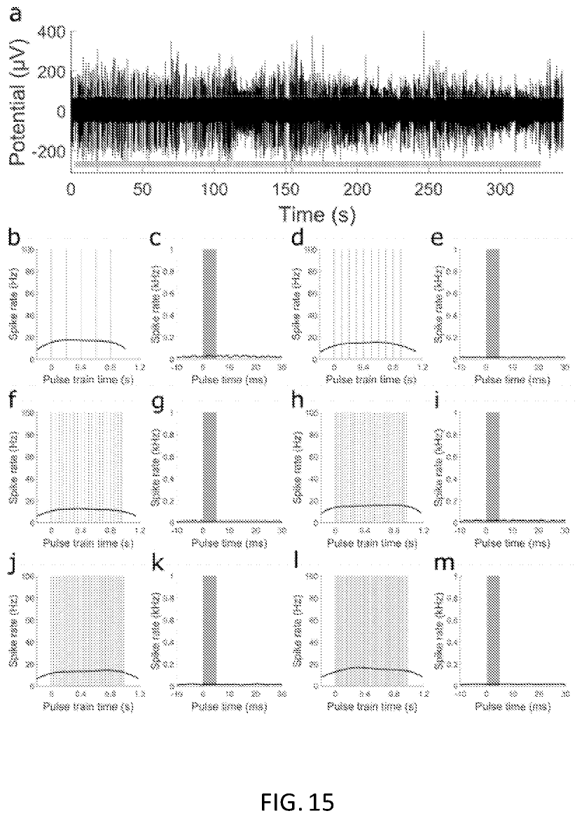

FIG. 15 shows EO-Flex optical testing as a function of different optical stimulation frequencies (5, 10, 20, 30, 40, and 50 Hz) in an awake mouse without light-activated protein expression. All other stimulation parameters were held constant (i.e., optical stimulation power, 200 μW; pulse width, 4.5 ms; on/off cycling, 1 Hz): (a) shows electrical recording (black) using a 50 Hz optical stimulus (blue). (b)-(m) show Bayesian adaptive kernel smoother (BAKS) estimation for the firing rate over the pulse train window (left) and the BAKS firing rate for the individual pulses (right) for an optical stimulation frequency of 5 Hz (b-c), 10 Hz (d-e), 20 Hz (f-g), 30 Hz (h-i), 40 Hz (j-k), and 50 Hz (l-m).

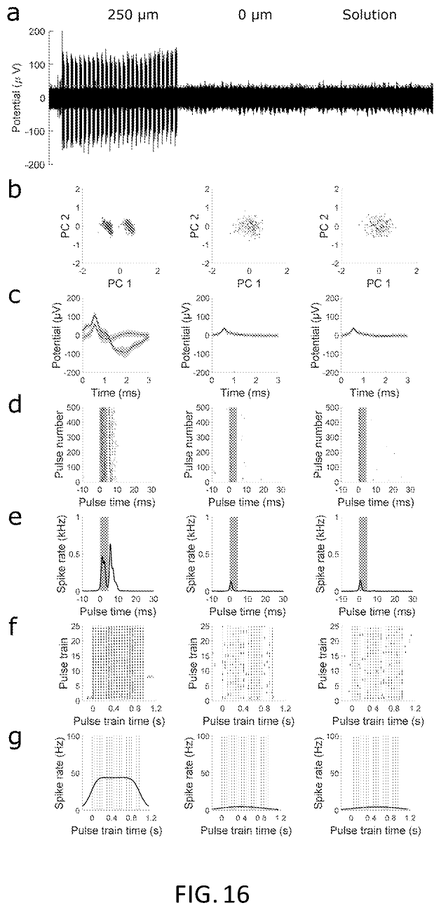

FIG. 16 shows EO-Flex testing in a live Thy1-ChR2-YFP mouse as a function of recording depth. All other stimulation parameters are held constant (optical stimulation power, 208 μW; stimulation frequency, 20 Hz; pulse width, 4.5 ms; on/off cycling, 1 Hz). Recording depths are ˜250 μm, 0 μm (i.e., at the agarose/brain interface), and in the saline solution above the craniotomy. FIG. 16(a) shows an optically evoked activity at different depths. FIG. 16(b) shows the first two principal components (PCs) of respective electrical recordings plotted with the Calinski-Harabasz metric for determining the number of clusters for mixed Gaussian fitting. FIG. 16(c) shows an average waveform (solid line) for each cluster from FIG. 16(b) with the shaded region representing one standard deviation. FIG. 16(d) shows peri-stimulus plots for all optical pulses with spikes color-coordinated with the cluster from which they come. FIG. 16(e) shows Bayesian adaptive kernel smoother (BAKS) estimation of the firing rate over the short time window around the optical pulses. FIG. 16(f) shows peri-stimulus plot for each optical pulse train for the different recording depths. FIG. 16(g) shows BAKS estimation for the firing rate over the pulse train window.

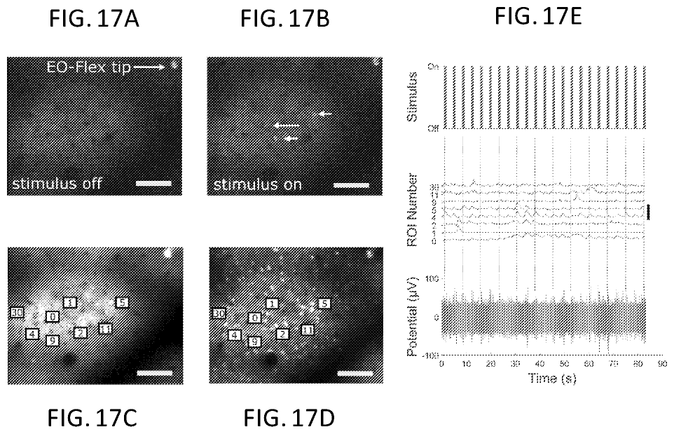

FIGS. 17A-17E show in vivo calcium imaging combined with simultaneous electrical recordings in the cortex of AAV2-CaMKII-C1V1-mCherry injected Vglut2-GCaMP6f mice confirms EO-Flex mediated optical excitation of neurons. FIGS. 17A-17B show fluorescence images from a time-lapse recording showing green fluorescent calcium indicator expressing neurons in layer 2/3 (depth, 270 μm) of Vglut2-GCaMP6f transgenic mice before FIG. 17A and immediately after FIG. 17B optical pulse train delivery to AAV2-CaMKII-C1V1-mCherry transduced cells. The probe tip is visible in the upper right corner of the field of view. The arrows in FIG. 17B indicate neurons that responded with fluorescence calcium transients to the optical pulses from the EO-Flex probe. Scale bar is 60 μm. FIG. 17C shows an average fluorescence image showing GCaMP6f expressing cells overlaid with regions of interest (ROIs) active during the 80 s stimulation period. Active ROIs are identified by automated analysis of cellular calcium signals using Suite2p. Scale bar is 60 μm. FIG. 17D shows an average fluorescence image showing AAV2-CaMKII-C1V1-mCherry transduced neurons within the same field of view as in FIG. 17C, confirming opsin expression in cells that show time-locked calcium responses to delivered optical pulses. Scale bar is 60 μm. FIG. 17E shows an example of successful optical excitation of neural activity is confirmed by simultaneous electrical recordings with the EO-Flex probe. Specifically, FIG. 17E shows at top delivered optical pulses (stimulation frequency, 8 Hz; pulse width, 12.2 ms; on/off cycling, 1 Hz) using 600 μW of power at the probe tip, and at center calcium transients within the individual ROIs indicated in FIG. 17C (Scale bar represents 2 dF/F), and at bottom recorded multi-unit activity after eliminating Becquerel effect mediated artifacts, caused by transient scanning of the imaging beam across the probe tip. Dashed lines are added for alternating pulse trains to aid in visual correlation between delivered optical pulses and measured calcium spiking and electrical activity.

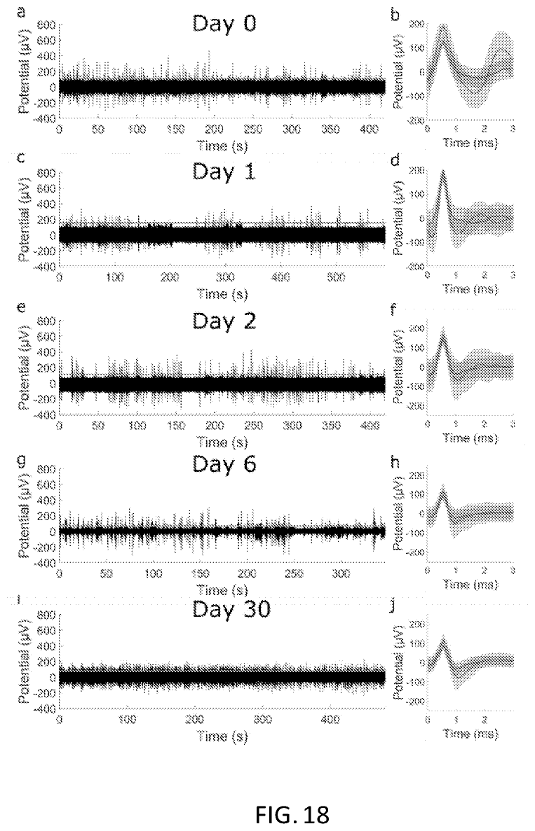

FIG. 18 shows chronic recordings with EO-Flex probes. In FIG. 18, (a)-(j) show spontaneous activity (left) and corresponding spike-sorted average waveforms with shaded one standard deviation (right) acquired with the same EO-Flex probe at different time points up to 1 month after implantation into the barrel cortex.

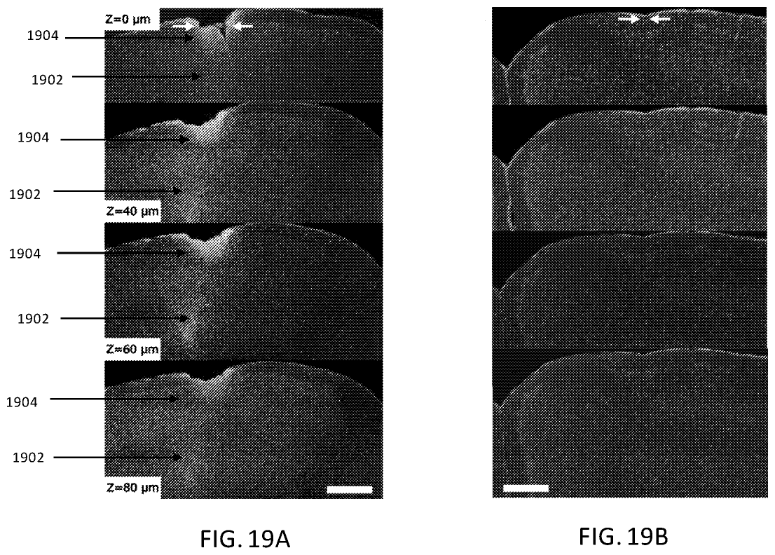

FIGS. 19A-19B show EO-Flex probes evoke minimal tissue inflammatory responses compared to multimode fibers commonly used in optogenetic experiments. Specifically, FIGS. 19A-19B show example images of 20 μm thick serial coronal brain sections around the multimode fiber (FIG. 19A) and EO-Flex probe (FIG. 19A) implantation sites (boundaries indicated by white arrows). Both the multimode fiber (diameter, 250 μm) and EO-Flex probe (diameter, 12 μm) are advanced to ˜1 mm depth into the cortex. Images are taken one week after brain implantation in Cx3cr1 GFP/+ mice with labeled microglia (1902). The sections are co-stained with anti-NeuN (blue) and anti-GFAP (1904) antibodies to label neurons and astrocytes, respectively. z denotes slice spacing in microns. Scale bars are 400 μm.

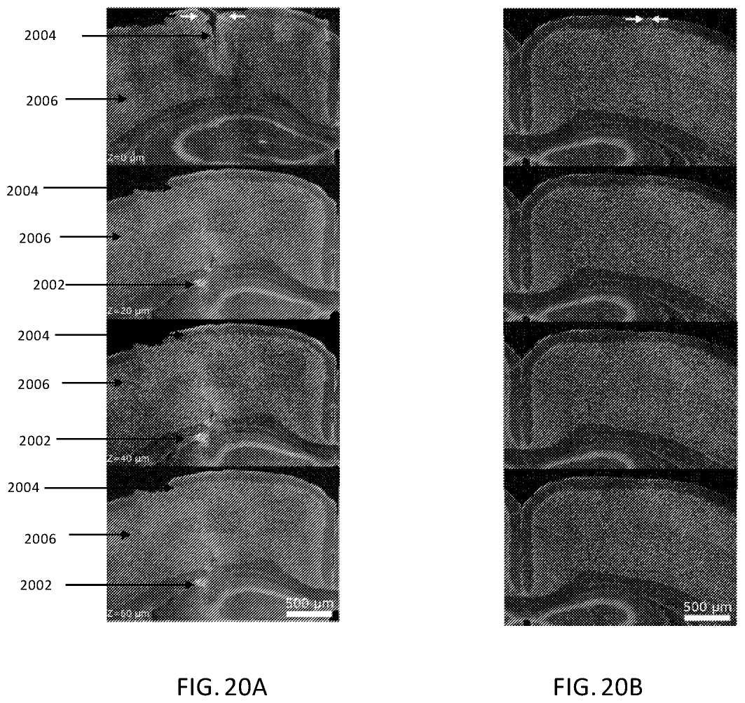

FIGS. 20A-20B show EO-Flex probes evoke minimal tissue inflammatory responses at 30 days post implant when compared to larger multimode fibers commonly used in optogenetic experiments. FIGS. 20A-20B show Example images of 20 μm thick serial coronal brain sections around the multimode fiber (FIG. 20A) and EO-Flex probe (FIG. 20B) implantation sites (boundaries indicated by white arrows). Both the multimode fiber (diameter, 250 μm) and EO-Flex probe (diameter, 12 μm) were advanced to ˜1 mm depth in the cortex. Images were taken one month after implantation in heterozygous Cx3cr1-GFP mice with labeled microglia (2002). The sections were co-stained with anti-NeuN (2006) and anti-GFAP (2004) antibodies to label neurons and astrocytes, respectively. z denotes slice spacing in microns. Results were reproduced across two different animals at 30 days post implant.

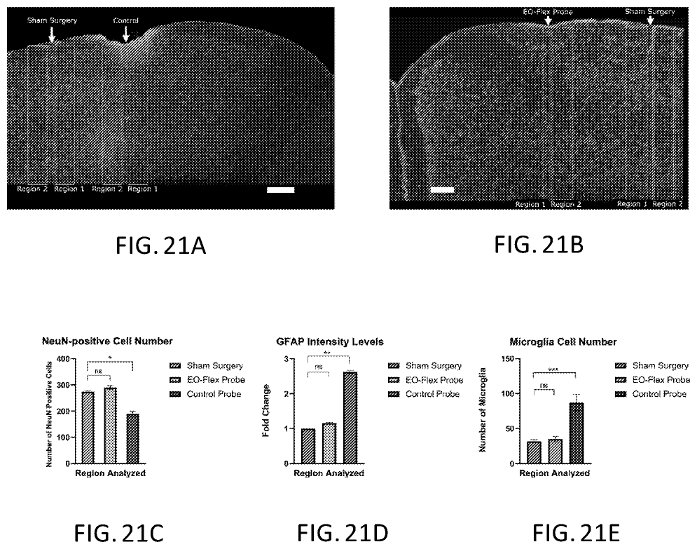

FIGS. 21A-21E show EO-Flex probes evoke minimal tissue responses compared to multimode fibers commonly used in optogenetic experiments. FIGS. 21A-21B show example images (from FIGS. 21A-21B) showing the analysis approach. Cellular responses are quantified and averaged across two 150 μm×1 mm analysis regions (white boxes) flanking each insertion site. To distinguish surgery from probe related tissue responses, an additional craniotomy of comparable size is made 0.7 mm lateral to each device implantation site. The same analysis approach is used to quantify cellular responses at this sham surgery site. Scale bar 200 μm and 150 μm respectively. FIG. 21C shows population analysis showing the impact of multimode fiber or EO-flex probe implantation on neuronal cell numbers. FIG. 21D shows population analysis showing the impact of multimode fiber or EO-flex probe implantation on astrocyte reactivity, as measured by GFAP expression level. FIG. 21E shows population analysis showing the impact of multimode fiber or EO-flex probe implantation on microglia reactivity, as measured by microglial cell number.

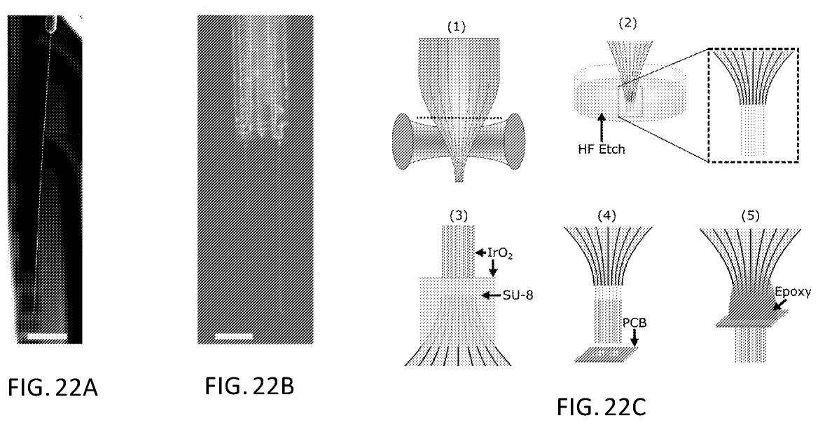

FIGS. 22A-22C show scaling strategies of the EO-Flex probes for both probe length and arrays. FIG. 22A shows two electron micrographs overlaid to show a 3.7 mm probe using a longer microfiber core. Scale bar 500 μm. FIG. 14B shows a 3×1 EO-Flex array with individually addressable optical channels (442 nm and 543 nm output). Scale bar is 250 μm. FIG. 22C shows a proposed method for scaling probes into large two- or three-dimensional arrays (number of probes >100): (1) Utilize the same heat and pull strategy used to fabricate fiber bundles. Cut bundle at a desired backend diameter near the dashed line; (2) Use an acid bath to etch the surrounding cladding on the individual fiber cores with the length determined by the desired insertion depth; (3) Invert etched structure and mask the bottom portion of the device to ensure electrical channels remain separate, then deposit desired metal cladding layer(s); (4) Insert the array into a printed circuit board (PCB) with via spacings tuned for the desired probe spacing/density that also allows tolerance for the insertion process (e.g., ˜50 μm); and (5) After inserting the array into the PCB, individual electrical connections are made using for example pin-in-paste reflow soldering techniques. The final assembly would use an adhesive to form a stable mechanical interface between the fiber bundle and PCB. After which point the PEDOT:PSS and Parylene-C could then be deposited as previously described.

In some implementations, EO-Flex probes are fabricated using micro- and nanofiber optical cores (see Methods). In one example, mass-producible silica microfibers can be used as the core that enables probes with lengths surpassing 3 mm while maintaining a diameter of <12 μm (FIGS. 22A-22C). However, the fabrication protocol is general and can be used with other optical cores, including subwavelength metal oxide nanofiber waveguides to produce ultra-miniaturized probes (FIGS. 8A-8C). In order to enable efficient coupling to optogenetic hardware, the microfibers are first placed on a silicon substrate, with one end of the fiber protruding the edge of the substrate, and then butt-coupled to a cleaved SMF (FIG. 1A). In some implementations, an active alignment can be used to maximize mode overlap between the microfiber and SMF. The coupling is locked in using a UV-curable optical adhesive droplet on the end of the SMF (FIG. 1B).

In some embodiments of the disclosed technology, to create a robust detachable interface for in vivo testing, the SMF is inserted into a ceramic ferrule. The distal end of the ferrule assembly is machine polished to allow coupling to a patch cable (FIGS. 1G-1H). Other interface designs for different applications are conceivable (FIG. 22).

In some embodiments of the disclosed technology, to form a low noise conductive layer around the probe tip, a 379±43 nm layer of iridium oxide (IrOx) is sputtered on the microfibers followed by a 362±137 nm electrochemically deposited layer of poly(3,4-ethylene dioxythiophene) polystyrene sulfonate (PEDOT:PSS) (FIG. 1C). The porous nature of IrOx allows better adhesion of the conductive PEDOT:PSS layer and enhances the overall electrical performance of the probe. The probe is passivated with 1.76±0.16 μm of Parylene-C to electrically isolate the probe and provide a biocompatible surface (FIG. 7). In order to expose the electrical and optical surfaces, a focused ion beam is used to cleave off the tip (FIG. 1E; see Methods). FIG. 1G shows the final probe design and FIG. 1H shows a photograph of a completed probe. Through the combination of IrOx and PEDOT:PSS, electrical impedances of <1 MΩ at 1 kHz are achieved from electrode areas of <15 μm2 (FIG. 1F).

The probes' optical properties are first assessed by imaging the output cone angle in a dye solution (FIG. 1D, inset), which shows a uniform output with a divergence angle of 10-15°. In some implementations, after the cladding layers are placed on the probe, no detectable scattering light is observed from the microfiber/SMF interface (FIG. 1D) as compared to the pre-cladding probe (FIG. 1B). The optical losses between a laser-coupled patch cable and the EO-Flex output are quantified using three different wavelengths (473 nm, 543 nm, 600 nm) with all devices showing <7 dB (n=4). These values match up well with simulated results for an ˜2 μm mode misalignment in the ferrule sleeve (FIGS. 6A-6G). Electrochemical impedance spectroscopy (EIS) is performed on the probes while submersed in a 1× phosphate-buffered solution (PBS). All probes fabricated and tested show an average electrical impedance of 844±179 kΩ at 1 KHz (n=4; FIG. 1F; FIGS. 7A-7D) after cladding deposition and milling the tip, compared to >10 MΩ before PEDOT deposition.

In some embodiments of the disclosed technology, to confirm that EO-Flex probes allow high-sensitivity electrical measurements in vivo, simultaneous extracellular recordings and two-photon imaging in the cortex of isoflurane-anesthetized mice can be performed. Imaging of fluorescently labeled cells (see Methods) enabled the monitoring of insertion and targeted movement of the probe through the tissue (FIG. 2A; FIGS. 17A-17E). Probes readily penetrated the exposed dura with minimal buckling when using water immersion and reached target regions in optically accessible cortical layer 2/3. When mounted to a three-axis micromanipulator, fine adjustment of the probe tip position, once inside the tissue, is feasible to optimize the signal-to-noise ratio and target individual neurons. However, the lateral movement is typically limited to <30 μm. Using this approach, endogenous multi- and single-unit activity (FIG. 2B) can be acquired. Principle component analysis (PCA) and Gaussian clustering of electrical recordings are used to determine the number of distinct units (FIG. 2C). Spiking rates are calculated using a Bayesian Adaptive Kernel Smoother (BAKS) algorithm applied to the full duration of the recording (FIG. 2D). FIGS. 2B-2E show a representative recording. Using an ˜1 mm-long EO-Flex probe, electrical recordings can be obtained from deeper cortical areas up to the probes' maximum length. The electrical signature of these recordings suggests that all cortical layers can be accessed (FIG. 9).

To demonstrate the EO-Flex probes' ability to record peripherally evoked activity, we inserted them into the barrel cortex, which receives sensory input from whiskers on the opposite side of the body. While advancing the probe into the brain, we periodically deflected the whiskers using air puffs while the animal was still under isoflurane anesthesia. Once the correlated activity was observed, probe position (˜900 μm insertion) was locked in place and anesthesia was turned off. Measurements commenced 30-60 min after the animals began to walk. Video recordings were used to verify air puff-mediated whisker deflections and record spontaneous whisking behavior under infrared illumination (FIGS. 3A-3C). Additionally, mouse locomotor activity was recorded using an optical encoder attached to the spherical treadmill on which the animal was positioned. Whiskers were deflected using various pulse frequencies (2, 3, and 5 Hz) and widths (20 and 50 ms) (FIGS. 3F-3Q), leading to stimulus-dependent increases in spike rate, which were most pronounced during periods of rest (i.e., in the absence of spontaneous whisking). To further corroborate the probe's positioning in the barrel cortex, we conducted EO-Flex probe-mediated electrical stimulations (0-300 μA biphasic pulses at 100 Hz), resulting in current amplitude-dependent whisker deflections (FIGS. 10A-10D).

To demonstrate the EO-Flex probe's ability to optically evoke neural activity while simultaneously electrically recording with the same probe, experiments can be performed in anesthetized Thy1-ChR2-YFP mice with blue light-activated ion channel Channelrodopsin-2 (ChR2) expression in neurons. Probes are again inserted into cortical layer 2/3 under visual control. A 473 nm diode-pumped solid-state (DPSS) laser, suitable for exciting ChR2, is coupled into the probe, and stimulation parameters are swept systematically (e.g., FIGS. 11, 13, 14, 16). The stimulation frequency (10 Hz-60 Hz), pulse width (0.6-9.8 ms), and output power (5-208 μW) may be varied to determine the optimal settings to excite ChR2-expressing neurons. Using waveform analysis on simultaneously recorded electrical activity, a minimum power of 29 μW (2,849 mW mm-2) is required for firing of the cells in sync with the optical pulse train (FIG. 11). In the example recording shown in FIGS. 4A-4B, PCA combined with a mixed Gaussian fit for the clustering of the data yielded two primary clusters (FIG. 4C) with two different waveforms (FIG. 4D) occurring during the stimulation period (FIGS. 4E-4G). There is minimal interference (e.g., Becquerel effect) between the proximal optical and electrical channels, as demonstrated by retracting the EO-Flex probe away from ChR2-expressing neurons, or placing it in a buffer solution, while optically pumping at maximum power (208 μW) using the same optogenetic pulse trains (FIG. 16). At this maximum power, neural circuits responded with minimal temporal lag (FIG. 11(f)) and could follow frequency stimuli up to 40 Hz before struggling to maintain synced firing (FIG. 14).

The ability of EO-Flex probes to optically evoke neural activity is further verified by two-photon calcium imaging in Vglut2-GCaMP6f mice. Four to five weeks after the cortical injection of an AAV2-CaMKII-C1V1-mCherry vector (see Methods), expressing the green light-activated ion channel C1V1 in neurons, probes are inserted into layer 2/3 regions with C1V1 expression (FIGS. 17A-17E). A 556 nm DPSS laser is coupled to the EO-Flex probe, and stimulation parameters are swept while simultaneously monitoring neuronal calcium transients. Delivered optical pulses led to correlated calcium spiking in C1V1-positive neurons within the field of view (FIGS. 17A-17E). The successful optical evocation of neural activity is also verified by simultaneous electrical recordings (FIG. 17E). Together, our in vivo data demonstrate the ability of EO-Flex probes to electrically record and optically modulate neural activity in the intact brain.

The EO-Flex probes allow targeting and entraining of opsin-expressing cells at firing rates ranging from 10 to 50 Hz (FIG. 14). While the minimum power (29 μW) to reliably activate neurons is higher than in previous reports (1-10 mW mm-2), it should be noted that due to the EO-Flex probes' small optical core (3.6 μm), and anticipated light absorption and scattering from the tissue, higher intensities are expected to create illumination volumes that are large and strong enough to recruit neurons successfully compared to conventional larger core fiber optics. Monte Carlo simulations in FIG. 11(b) indicate that at a 29 μW stimulation power the optical power density drops below the optogenetic threshold of 1 mW mm-2 at around 1.2 mm from the tip. Even at the maximum stimulation power utilized in our optogenetic experiments (208 μW or 20,435 mW mm-2 at the probe tip), we did not observe any adverse cellular effects (e.g., sustained changes in firing rate or calcium levels) (FIGS. 15-17). Recent studies have suggested that continuous optical exposure with powers <0.25 mW results in no temperature effects on neural activity (i.e., degraded electrical signals over the stimulation period) 28. To further ensure minimal optical heating effects on the neural tissue, we utilized short pulse widths and optical powers of less than 250 μW (FIGS. 11-15). Minimal heating effects are expected when using these illumination parameters, which was verified by applying previously validated heating models to the EO-Flex probes (FIG. 12B).

The disclosed technology can be implemented in some embodiments to evaluate the brain's response to probe implantation. EO-Flex probes are implanted into the cortex of heterozygous Cx3cr1-GFP mice with labeled microglia for 6 and 30 days. A 250 μm-diameter multimode fiber, commonly used in optogenetic experiments, is inserted using the same stereotaxic coordinates but on the opposite hemisphere for comparison. Serial brain sections are prepared that included both implantation sites. Tissue slices are co-stained with anti-GFAP and anti-NeuN antibodies to quantify reactive astrogliosis and neuronal loss, respectively (n=4 mice; N=8 sections per mouse) (FIGS. 5, 19, 20). At day 6 after implantation, we found that the multimode fiber led to significant neuronal loss, a 2.08±0.23-fold increase in microglia numbers, and a 2.68±0.60-fold increase in GFAP levels (FIG. 5e-g). In contrast, the EO-Flex probes showed no significant decrease in NeuN-positive cells or increase in microglia numbers or GFAP levels around the insertion site (FIG. 5e-g). At day 30 after implantation, neuronal loss was again observed for the implanted control multimode fiber, as well as a 2.33±0.27-fold increase in microglia numbers, and a 2.81±0.63-fold increase in GFAP levels (FIGS. 5L-5N). In contrast, the EO-Flex probes showed no significant decrease in NeuN-positive cells or increase in GFAP levels, but a small increase in microglia numbers (FIGS. 5L-5N). These results indicate that tissue responses to EO-Flex probe insertion or movement during the implantation period are negligible at time points when inflammatory responses are typically most prominent, and considerably smaller compared to standard probes used for optogenetic experiments. Finally, given this minimal immune response, we also performed chronic recordings up to 30 days after EO-Flex probe implantation. These recordings revealed excellent signal-to-noise ratios across all investigated time points (day 0, 1, 2, 6, and 30) (FIG. 18).

The brain's response to probe implantation can be evaluated. EO-Flex probes are implanted into the cortex of Cx3cr1 GFP/+ mice with labeled microglia for seven days. A 250 μm-diameter multi-mode fiber, suitable for optogenetic experiments, is inserted using the same stereotaxic coordinates but on the opposite hemisphere for comparison. Serial brain sections are prepared that included both implantation sites. Tissue slices are co-stained with anti-GFAP and anti-NeuN antibodies to quantify reactive astrogliosis and neuronal loss, respectively (e.g., FIGS. 19, 21). In some embodiments of the disclosed technology, implantation of the multi-mode fiber is associated with significant neuronal loss (FIG. 21C), a 2.72±0.35 fold increase in microglia numbers, and 2.62±0.1 fold increase in GFAP levels (FIGS. 21D-21E). In contrast, the EO-Flex probes show no significant decrease in NeuN-positive cells or increase in microglia numbers and GFAP levels around the insertion site (FIGS. 21C-21E). Together, these results indicate that tissue responses to EO-Flex probe insertion and potential animal behavior-related probe movement during the implantation period are negligible at a time point when inflammatory responses are typically most prominent, and considerably smaller compared to standard probes used for optogenetic experiments.

EO-Flex probes allow targeting and entraining of opsin-expressing cells at firing rates ranging from 10 Hz to 30 Hz (FIG. 14). A minimum power threshold of 29 μW (2,849 mW mm-2 at the probe tip) is required for reliable activation of neurons, and while this irradiance is higher than in previous reports (1-10 mW mm-2), optical degradation effects in the tissue (FIGS. 17A-17E) are not observed. Monte Carlo simulations show that, at 29 μW stimulation power, the optical power density drops below the optogenetic threshold of 1 mW mm-2 at around 1.2 mm from the tip. Even at the maximum stimulation power utilized in our optogenetic experiments (208 μW or 20,435 mW mm-2 at the probe tip, which drops to 2, 168 mW mm-2 at a distance of 50 μm), any adverse cellular effects (e.g., sustained changes in firing rate or calcium levels) are not observed. Recent studies have suggested that continuous optical exposure with powers <0.25 mW result in no temperature effects on neural activity (i.e., degraded electrical signals over the stimulation period). To ensure minimal optical heating effects on the neural tissue, short pulse widths and optical powers of less than 250 μW (FIG. 11) may be utilized.

Developing probes that can reach deeper brain regions is straightforward with the developed fabrication protocols as virtually any microfiber length can be generated (FIG. 22A). However, for a given set of cladding layers, the probe's stiffness decreases with length. Therefore, longer probes might require additional tactics to overcome low buckling forces during the insertion process (e.g., dissolvable sugar coatings, or rigid polymer layers). Alternatively, a surgical incision in the dura could facilitate probe insertion. Regardless of probe length, our implantation studies demonstrated that the small-footprint EO-Flex probes have a drastically reduced immune response in comparison to standard multi-mode fibers.

In some embodiments of the disclosed technology, a novel multi-modal coaxial microprobes can be fabricated and such a multi-modal coaxial microprobes can demonstrate the ability to optically stimulate and electrically record from intrinsic neural circuits with minimal interference between the two modalities. The small footprint and high aspect ratio of the EO-Flex probes allow for minimally invasive interfacing with neural circuits. Further size reduction is possible with this coaxial design using smaller fiber optic cores, however, the tradeoff is an increase in optical losses and electrical impedance (FIGS. 8A-8C). Although the probes' capabilities are only tested in the brain, as a platform with excellent control over probe diameter and length, the choice of cladding materials with various chemical compositions, inherent mechanically flexibility, and a clear route to scaling up probe densities (e.g., translating the cladding deposition process to fiber bundles) (FIGS. 22A-22C), this technology should find immediate applications as minimally invasive interfaces in diverse nervous system regions, including the spinal cord and peripheral nerves.

The disclosed technology can be implemented in some embodiments to provide multi-modal coaxial microprobes and demonstrate their ability to optically stimulate and electrically record from intrinsic neural circuits with minimal interference between the two modalities. The small footprint and high aspect ratio of the EO-Flex probes allow minimally invasive interfacing with neural circuits. Further size reduction is possible with this coaxial design using smaller fiber optic cores; however, the tradeoff is an increase in optical losses and electrical impedance (FIGS. 8A-8C). Although the probes' capabilities were only tested in the brain, as a platform with excellent control over probe diameter and length, the choice of cladding materials with various chemical compositions, inherent mechanical flexibility, and a clear route to scaling up probe densities (e.g., translating the cladding deposition process to fiber bundles) (FIGS. 22A-22C), this technology should find immediate applications as minimally invasive interfaces in diverse nervous system regions, including the spinal cord and peripheral nerves.

Developing probes that can reach even deeper brain regions is straightforward with the developed fabrication protocols as virtually any microfiber length can be generated (FIG. 22A). However, for a given set of cladding layers, the probe's stiffness decreases with length. Therefore, longer probes might require additional tactics to overcome low buckling forces during the insertion process (e.g., dissolvable sugar coatings, or rigid polymer layers). Alternatively, a surgical incision in the dura could facilitate probe insertion. Regardless of probe length, our implantation studies demonstrated that the small-footprint EO-Flex probes have a drastically reduced immune response com-pared to standard multimode fibers.

### Methods

### Probe Fabrication

In some embodiments of the disclosed technology, silica microfibers (core and total diameters: 3.63±0.31 μm and 5.60±0.42 μm, respectively) with lengths varying between 500 μm and 1 cm are generated from leeched fiber optic bundles. After cleaving, individual fibers are dispersed onto a silicon substrate. A tungsten needle mounted on a three-axis micromanipulator is used to position the microfibers near a substrate edge with one end of the fiber being suspended >100 μm from the edge.

To enable efficient optical coupling of the waveguide to standard optogenetic hardware, a single-mode fiber (SMF) with a mode field diameter (2.8-3.4 μm) slightly smaller than the microfiber core is chosen. To create a robust detachable interface for in vivo testing, the SMF is inserted into a ceramic ferrule and secured in place with quick cure epoxy. The ferrule assemblies were then machine polished until a smooth coupling interface was observed through a fiber inspection scope, and the opposing fiber end (for coupling to the waveguide) was cleaved using a ruby scribe. The ferrule assembly was mounted on a three-axis stage, and the scribed end was maneuvered into a droplet of UV-cured optical adhesive until a small droplet formed at the end. Efficient coupling between SMF and micro-/nanofiber was achieved using active alignment under an upright optical microscope equipped with a 0.4 NA 20× objective after coupling a 544 nm He—Ne laser source into the SMF. After maximizing power coupling into the waveguide by translating the SMF, the NOA 81 adhesive was secured by exposing it to UV light (325 nm line from a HeCd laser) for a duration of 30 s while continuously moving the beam around the droplet.

Before depositing the metal layer, the probe assemblies are placed in a custom aluminum block holder to mask the bottom part of the ferrule where light is coupled into the assembly. This ensured that the optical coupling interface is masked during metallization. This block is placed on a rotating plate inside a sputtering chamber. A thin (<10 nm) adhesion layer of titanium (2.5 m Torr, 5 s, 200 W) is deposited, followed by a 300 nm thick layer of iridium oxide (IrOx) (12 mTorr, 15 min, 100 W, 5 sccm O2 flow) or 500 nm of platinum (Pt) (2.5 mTorr, 20 min, 200 W). Iridium oxide was chosen for its ˜3× higher charge-injection capacity compared to conventional platinum layers, and its porous nature, which increases the electrochemical surface area of the metal layer.

Together, these procedures yielded ferrule assemblies for repeatable mounting to an in vivo imaging and optogenetics setup (FIGS. 2A-2E). Alternative interface designs (e.g., for probe arrays) are shown in FIG. 22A-22C.

In some implementations of the disclosed technology, EO-Flex probes are fabricated using one of two waveguides as the optical core: a) silica microfibers (SiOx) (FIGS. 1 and 7), or b) single crystalline tin dioxide (SnO2) nanofibers (FIGS. 8A-8C).

In some implementations, the SnO2 nanofibers are synthesized using thermal evaporation of SnO powders at high temperatures according to published protocols. Ceramic combustion boats were loaded with 1-5 grams of tin monoxide powder and placed in a tube furnace. The system was pumped down to <1 m Torr as the furnace was turned on to 1000° C. At operating temperature, system pressures were typically around 300 mTorr. The system was allowed to run for an hour, after which the furnace was turned off, and the system was allowed to cool while the vacuum pump remained on. The combustion boat was then removed, and nanowires found on the boat's rim were transferred to a silicon substrate to facilitate coupling to a cleaved SMF (FIGS. 1, 8).

In some implementations, silica microfibers (core and total diameters: 3.63±0.31 μm and 5.60±0.42 μm, respectively) with lengths varying between 500 μm and 1 cm are generated from leeched fiber optic bundles. After cleaving, individual fibers are dispersed onto a silicon substrate. A tungsten needle mounted on a 3-axis micromanipulator is used to position the microfibers near a substrate edge with one end of the fiber being suspended >100 μm from the edge.

In some implementations, the SnO2 nanofibers are synthesized using thermal evaporation of SnO powders at high temperatures according to published protocols 1. Ceramic combustion boats are loaded with 1-5 grams of tin monoxide powder and placed in a tube furnace. The system is pumped down to <1 mTorr as the furnace is turned on to 1000° C. At operating temperature, system pressures are typically around 300 m Torr. The system is allowed to run for an hour, after which the furnace is turned off, and the system is allowed to cool while the vacuum pump remained on. The combustion boat is then removed, and nanowires found on the rim of the boat are transferred to a silicon substrate.

### PEDOT-PSS Deposition

To further lower the electrical impedance of the probes, a poly(3,4-ethylenedioxythiophene)-polystyrene sulfonate (PEDOT:PSS) layer was deposited on the IrOx. Probes are submersed (˜100 μm of the probe tip) into a 0.01 M solution of EDOT with 2.5 mg/ml of poly(sodium styrene sulfonate) (PSS). The electrochemical deposition is performed using a platinum wire counter electrode and an Ag/AgCl reference electrode connected to an electrochemical potentiostat operating in the galvanostatic mode set to run at a current of 200 nA for 5-30 s. This yielded a 362±137 nm thick polymer layer (FIG. 1E and FIGS. 7A-7E). The PEDOT-IrOx (or PEDOT-Pt) coated microfibers were then passivated with 1.5-2 μm of parylene-C using chemical vapor deposition.

### In Vitro Probe Characterization

A focused ion beam set to 5 nA at 30 k V is used to cleave off the end of the probe and expose the electrical and optical channels, revealing a final probe diameter of 8-12 μm for the micro-fiber cores. Electrochemical impedance spectroscopy (EIS) is carried out to determine probe impedance in a 1× phosphate-buffered saline (PBS) using the same reference and counter electrodes described above. Optical coupling efficiency was determined by measuring light output from a fiber optic patch cable using three light sources (e.g., 473 nm, 543 nm, and 673 nm) interchangeably coupled into the cable. Light power was measured by placing the ferrule 5-10 mm away from the detector head of a digital power meter. A ceramic ferrule sleeve is then slid halfway onto the patch cable, and different EO-Flex probes are slid into the opposite end to couple light through. Light power from the tip of the EO-Flex probes is measured using a similar protocol to the patch cable.

### Animal Subjects

For combined optogenetic and electrophysiological experiments, Thy1-ChR2-YFP male mice can be used, and for combined calcium imaging, optogenetics, and electrophysiological experiments, AAV2-CaMKII-C1V1-mCherry-injected Vglut2-GCaMP6f male mice can be used, and for immune response and all other studies, heterozygous Cx3cr1-GFP male mice can be used.

### Stereotactic Viral Vector Injection

Surgical procedures closely followed previously established protocols. Briefly, thin-wall glass pipettes are pulled on micropipette puller. Pipette tips are cut at an acute angle under 10× magnification using sterile techniques. Tip diameters were typically 15-20 μm. Pipettes that did not result in sharp bevels nor had larger tip diameters were discarded. Millimeter tick marks were made on each pulled needle to measure the virus volume injected into the brain.

Mice are anesthetized with isoflurane (4% for induction; 1-1.5% for maintenance) and positioned in a computer-assisted stereotactic system with digital coordinate readout and atlas targeting. Body temperature was maintained at 36-37° C. with a DC temperature controller, and ophthalmic ointment was used to prevent the eyes from drying. A small amount of depilator cream was used to remove hair at the designated skin incision site. The skin was cleaned and sterilized with a two-stage scrub of betadine and 70% ethanol. A midline incision was made beginning just posterior to the eyes and ending just passed the lambda suture. The scalp was pulled open, and the periosteum was cleaned using a scalpel and forceps to expose the desired hemisphere for calibrating the digital atlas and coordinate marking. Once reference points (bregma and lambda) are positioned using the pipette tip and entered into the program, the desired target is set on the digital atlas. The injection pipette is carefully moved to the target site (coordinates: AP-1.5 mm, ML 1.5 mm). Next, the craniotomy site is marked, and an electrical micro-drill with a fluted bit (0.5 mm tip diameter) is used to thin a 0.5-1 mm diameter part of the bone over the target injection site. Once the bone is thin enough to flex gently, a sterile 30 G needle with an attached syringe is used to carefully cut and lift a small (0.3-0.4 mm) segment of bone.

For injection, a drop of the virus is carefully pipetted onto parafilm (1-2 μl) for filling the pulled injection needle with the desired volume. Once loaded with sufficient volume, the injection needle is slowly lowered into the brain until the target depth (DV 0.2 mm) is reached. Manual pressure is applied using a 30-mL syringe connected by shrink tubing, and 0.4 μl of the AAV2-CaMKII-C1V1-mCherry vector (6.1E+12 VP/mL; undiluted) is slowly injected over 5-10 min. Once the virus is injected, the syringe's pressure valve is locked. The position is maintained for ˜10 min to allow the virus to spread and to avoid backflow upon needle retraction. Following the injection, the skin is sutured along the incision. Mice are given subcutaneous Buprenex SR (0.5 mg per kg) and allowed to recover before placement in their home cage. The vector is allowed to express for 4-5 weeks before in vivo recordings.

### Animal Preparation for In Vivo Recordings

Surgical procedures closely follow established protocols. In some implementations, mice are anesthetized with isoflurane (4-5% for induction; 1-1.5% for maintenance) and implanted with a head plate on a custom surgical bed. Body temperature is maintained at 36-37° C. with a DC temperature control system and ophthalmic ointment is used to prevent the eyes from drying. Depilator cream is used to remove hair at the designated skin incision site. The skin is thoroughly cleansed and disinfected with a two-stage scrub of betadine and 70% ethanol. A scalp portion is surgically removed to expose frontal, parietal, and interparietal skull segments. Scalp edges are attached to the skull's lateral sides using a tissue-compatible adhesive. A custom-machined metal plate is affixed to the skull with dental cement, allowing the head to be stabilized with a custom holder. Mice are given Buprenex SR (0.5 mg/kg) before relief from anesthesia and allowed to recover for at least three days before further preparation.

For combined imaging and electrophysiological recordings, an ˜2 mm×4 mm diameter craniotomy was performed over the target area (e.g., the AAV vector injection site). The dura mater overlying the cortex was kept intact. Tissue motion was controlled by covering the exposed tissue with a 1% agarose solution and coverslip. The coverslip is affixed to the skull. To enable probe entry into the cortex, the agarose and coverslip were cut on one side to be flush with the craniotomy. Recordings started immediately after optical window preparation. The depth of anesthesia was monitored throughout the experiment and adjusted as needed to maintain a breath rate of approximately 55-65 breaths per minute. Saline was supplemented as needed to compensate for fluid loss.

For electro-optical measurements under awake conditions, mice were first habituated to head restraint on a spherical treadmill (typically three sessions, 30-90 min/session, one session/day on three consecutive days). Following habituation, an ˜0.3-0.5 mm diameter craniotomy was performed over the target area (barrel cortex; coordinates: AP-1.0 mm, ML 3.0 mm) under general anesthesia. Mice are then transferred to the spherical treadmill and allowed to recover from anesthesia for at least 30-60 min, depending on the duration they had spent under anesthesia. Following electro-optical measurements, the probe is locked into position by first covering the probe/tissue interface with 1% agarose solution and then applying an adhesive and dental cement, thereby affixing the ferrule to the skull. Mice are allowed to recover in their home cage before subsequent recordings on different days.

In some implementations of the disclosed technology, surgical procedures may follow established protocols. For example, mice are anesthetized with isoflurane (4-5% for induction; 1%-1.5% for maintenance) and implanted with a head plate on a custom surgical bed. Body temperature is maintained at 36° C.-37° C. with a DC temperature control system, and ophthalmic ointment is used to prevent the eyes from drying. Depilator cream is used to remove hair at the designated skin incision site. The skin is thoroughly cleansed and disinfected with a two-stage scrub of betadine and 70% ethanol. A scalp portion is surgically removed to expose frontal, parietal, and interparietal skull segments. Scalp edges are attached to the lateral sides of the skull using a tissue-compatible adhesive. A custom-machined metal plate is affixed to the skull with dental cement, allowing the head to be stabilized with a custom holder. An approximately 2 mm×4 mm diameter craniotomy is made over the target area (e.g., AAV injection site). The dura mater overlying the cortex is kept intact. A 1% agarose solution and coverslip are applied to the exposed cortical tissue. To facilitate probe entry into the tissue, the agarose and coverslip are cut on one side to be flush with the craniotomy, allowing direct cortical access through the agarose. The coverslip is affixed to the skull with dental cement to control tissue motion. Recordings commenced immediately after optical window preparation. The depth of anesthesia is monitored throughout the experiment and adjusted as needed to maintain a breath rate of approximately 55-65 breaths per minute. Saline is supplemented as needed to compensate for fluid loss.

### In Vivo Electrophysiology

To characterize the EO-Flex probes' electro-physiological properties in vivo, extracellular single- and multi-unit recordings are performed in the cortex of isoflurane-anesthetized and awake mice. The EO-Flex probes' electrical channel is connected to the positive terminal of a high impedance head stage using a custom adapter, whereas the negative terminal and ground is connected to an Ag/AgCl wire inserted above the cerebellar cortex. The adapter consisted of a ceramic block with an embedded patch cable end and removable copper clip soldered to a single core head stage wire. EO-Flex probes are mated with this adapter by sliding a ferrule sleeve onto the patch cable end, sliding the probe into this assembly, and then lowering the copper clip to contact the metal layer on the EO-Flex ferrule.

To allow targeted tissue insertion and precise positioning of the probe, the adapter is mounted to a motorized micromanipulator oriented at a defined angle with respect to the skull (e.g., ˜60 degrees for combined imaging and electrophysiology, and ˜0 degrees for measurements without imaging). After positioning the tip of the EO-Flex probe near the edge of the craniotomy, a few drops of physiological saline are pipetted onto the skull opening to facilitate mechanical insertion through the tissue interface (FIG. 2A).

Precise positioning can be aided by passing the differential amplifier's output through a speaker to serve as auditory feedback for probe proximity to active cells. The raw electrode signal is amplified, filtered (low cut-off, 300 Hz; high cut-off, 5 kHz; gain, 1000×), digitized (20 kHz), and stored on disk for off-line analysis. Positioning of the probe's tip near neuronal cell bodies is further aided by visual feedback in experiments involving imaging in fluorescent indicator-expressing transgenic mice.

Electrical stimulation (FIGS. 10A-10D) involved EO-Flex probe-mediated current pulse delivery (0 to 300 μA amplitude, 100 Hz stimulation frequency, 0.2 ms pulse width, 1 Hz stimulation period) with an isolated pulse stimulator connected to a function generator.

In some implementations of the disclosed technology, to characterize the electrophysiological properties of the EO-Flex probes, extracellular single- and multi-unit recordings in the cortex of isoflurane-anesthetized mice may be performed. The EO-Flex probes' electrical channel is connected to the positive terminal of a high impedance head stage (e.g., microelectrode AC amplifier) with the negative terminal and ground attached to an Ag/AgCl wire inserted above the cerebellar cortex. To allow targeted tissue insertion and precise positioning the probe is mounted to a motorized micromanipulator angled at approximately 30 degrees relative to the optical axis of the microscope. After positioning the tip of the EO-Flex probe near the edge of the craniotomy, a few drops of physiological saline are pipetted onto the exposed agarose/cortex interface to facilitate mechanical insertion through the agarose and dura (FIG. 2A). The probe tip is positioned near neuronal cell bodies in upper cortical layers (mostly layer 2/3) of fluorescent indicator-expressing transgenic mice using two-photon imaging. Precise positioning is aided by passing the output from the differential amplifier through a speaker to serve as auditory feedback for probe proximity to active cells. The raw electrode signal is amplified, filtered (low cut-off, 300 Hz; high cut-off, 5 kHz; gain, 1000×), digitized (20 kHz), and stored on disk for off-line analysis.

### Whisker Stimulation

The barrel cortex in a given hemisphere receives sensory input from whiskers located on the opposite side of the body. To deflect whiskers contralateral to the probe's recording location, we delivered air puffs with a micropipette connected through plastic tubing to a function generator-controlled pressure system. The function generator also operated an infrared LED positioned outside the animal's but within the video camera's field of view for synchronizing the analog and video data. The micro-pipette was connected to a motorized micromanipulator, allowing precise control over the whiskers being stimulated. Air pressure was directed away from the skin and eye and delivered in rostra-caudal direction. Air puff stimuli consisted of 2 s “on” followed by 2 s “off”, with varying pulse frequencies (2-5 Hz) and widths (20-100 ms).

### Two-Photon Microscopy

In vivo imaging is performed by utilizing a movable objective microscope equipped with a pulsed femtosecond Ti:Sapphire laser, two fluorescence detection channels; dichroic beam splitter; photomultiplier tubes, and image acquisition software. The laser excitation wavelength is set to 920 nm. The average laser power is <10-15 mW at the tissue surface and adjusted with depth to compensate for signal loss due to scattering and absorption. A 16×0.8 NA or 40×0.8 NA water-immersion objective was used for light delivery and collection. Spontaneous and optically evoked calcium activity is recorded in optical planes near the probe tip (frame rate, 8.14 Hz). To minimize the Becquerel effect mediated artifacts in electrical recordings, the imaging laser power is kept to a minimum. To record optically evoked calcium transients in optogenetic experiments, the image frame rate is synchronized with optical pulse train delivery and adjusted the phase such that regions in front of the probe tip are scanned when the DPSS laser is off (FIGS. 17A-17E).

### In Vivo Optogenetics

To excite ChR2 or C1V1, respectively, the light from a 200-mW 473 or 556 nm DPSS laser, directly modulated by an external function generator signal, is coupled into the probe. Light coupling into the probe is achieved by sliding the polished end of the ferrule into a ceramic sleeve and then sliding it onto the end of a custom fiber patch cable. Each stimulation trial lasts around 60 s, with the initial 5-10 s designated for recording spontaneous activity before the optical pulse train is delivered (stimulation power, 6-208 μW; pulse width, 0.6-9.8 ms; stimulation frequency, 10-50 Hz; duration, 1 s; inter-stimulus-interval, 1 s between pulse trains).

### Tissue Response Assessment

Heterozygous Cx3cr1-GFP mice with labeled microglia are implanted with an EO-Flex probe and a 250 μm-diameter multimode fiber suited for optogenetic deep brain stimulation on opposite hemispheres (+1.45 mm from midline). For implantation, an electrical micro-drill with a fluted bit (0.5 mm tip diameter) is used to thin a 0.5-1 mm diameter part of the bone. Once the bone is thin enough to flex gently, a sterile 30 G needle with an attached syringe was used to carefully cut and lift a small (0.3-0.4 mm) segment of bone. The probe or multimode fiber is advanced through this opening under visual control to a depth of approximately 1 mm using a computer-assisted stereotactic system. Dental cement is used to secure the devices in place. The firm bonding of the dental cement to the skull is facilitated by scarifying it with a bone scraper. To distinguish surgery from probe related tissue responses, additional craniotomies 0.7 mm lateral from the device implantation sites can be performed (FIGS. 19A-19B). To assess tissue inflammatory responses, mice are sacrificed 6 or 30 days after device implantation using CO2 asphyxiation. Transcardial perfusion is performed with 10% sucrose in PBS, followed by freshly prepared 4% PFA in PBS. Both hemispheres were post-fixed in 4% PFA in PBS overnight and subsequently infiltrated in 30% sucrose in PBS for one day and flash frozen in a TBS tissue freezing medium. The implanted hemispheres are coronally cryo-sectioned at 20 μm, air-dried overnight, and subsequently processed for staining. Sections are incubated overnight at 4° C. with primary antibody diluted in blocking buffer, then washed in PBS 0.1% Tween-20, and incubated for two hours at 22-24° C. in the dark with fluorophore-coupled secondary antibodies. Sections are washed, sealed with mountant, and stored at 4° C. Primary antibodies included anti-GFAP and anti-NeuN. Secondary antibodies (1:100) included Alexa Fluor 405 goat anti-rabbit and Alexa Fluor 633 goat anti-mouse. Confocal imaging of stained tissue sections is performed on software. Three-channel tiled z-stacks were acquired to produce images of whole-tissue sections (FIGS. 5, 19, 20). Image size is 1024×1024 pixels stitched into 3-5×3-5 tiles.

### Data Processing and Statistical Analyses

Neural activity is considered a spike if its amplitude crosses a threshold determined by:

$$Threshold = 4 * median ( \frac{\vert Recording \vert}{0.675} ) .$$

All observed spikes are then sorted according to the first two principal components into clusters using a mixed Gaussian fitting with the number of clusters optimized according to the Calinksi-Harabasz metric for cluster analysis calculated in MATLAB. Average firing rates are calculated using the Bayesian Adaptive Kernel Smoother (BAKS). Monte Carlo simulations are used to determine the propagation and illumination volume of the EO-Flex probe at different powers (FIG. 11).

Optogenetic heating profiles are created using previous models utilizing the Pennes bio-heat equation. Simulation parameters are for an EO-Flex probe optical radius of 1.8 μm, a wavelength of 470 nm, power of 1 mW, or 208 μW, and a cylindrical radius of 10 μm for temperature averaging in the time-based simulations (FIGS. 12A-12D).

In some implementations, peristimulus plots correlating optical stimuli with spiking events are calculated using kernel bandwidth optimization, which has been shown to accurately estimate the underlying spiking rate (FIG. 9(b)).

In some implementations, peristimulus plots correlating optical stimuli with spiking events are calculated using kernel bandwidth optimization, which has been shown to accurately estimate the underlying spiking rate (FIG. 4B). For stimulation frequencies of 10, 20, and 30 Hz, estimated firing rates closely following input frequencies is observed when accounting for the echo after stimulation. In comparison, frequencies of 40 and 50 Hz initially tracked the input frequency for the first couple of light pulses, but eventually, the firing rate decreased as the optical train progressed (FIG. 14(g)).

Analysis of the two-photon calcium imaging data is performed (FIGS. 17A-17E). Optically evoked calcium spiking is observed in optical planes near the probe tip. The disclosed analysis focuses on the optical planes in which at least three cellular-size regions of interest (ROIs) consistently responded throughout the stimulation period.

Immunostaining data are processed, analyzed, and plotted using software. All data are represented as mean±s.e.m. Group sample sizes are chosen based on previous studies and power analysis. Two-tailed paired t-tests determined P values. The following convention is used to indicate P values: “ns” indicates P>0.05, “*” indicates 0.01<P<0.05, “**” indicates 0.001<P≤0.01, and “***” indicates 0.0001<P≤0.001.

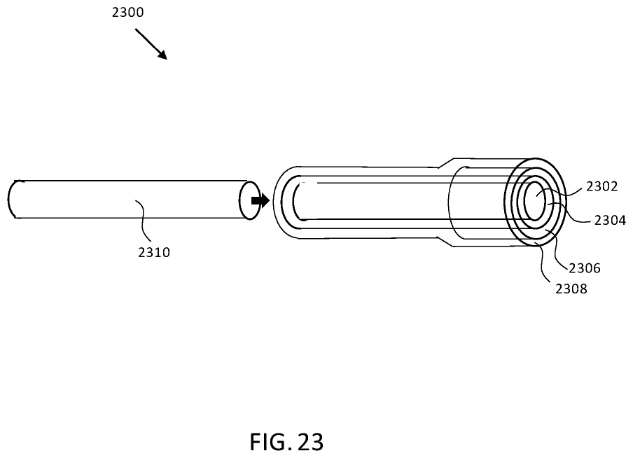

FIG. 23 shows an example of an electro-optical microprobe based on some embodiments of the disclosed technology.

In some implementations, an electro-optical microprobe 2300 includes an optical waveguide 2302 (e.g., an optical fiber) including first and second ends and a side surface between the first and the second ends, a first layer 2304 including a first electrically conductive material disposed over the side surface of the optical waveguide, a second layer 2306 including an electrically conductive polymer disposed on a portion of the first layer proximate to the first end of the optical waveguide, and an isolation layer 2308 including an electrically insulative material disposed the second layer and a remaining portion of the first layer that is not covered by the second layer.

In some implementations, the electro-optical microprobe 2300 further includes a single-mode fiber 2310 optically coupled to the second end of the optical waveguide.

In some implementations, the electro-optical microprobe 2300 further includes an adhesion layer (not shown) including a second electrically conductive material disposed over the side surface of the optical waveguide and below the first layer.

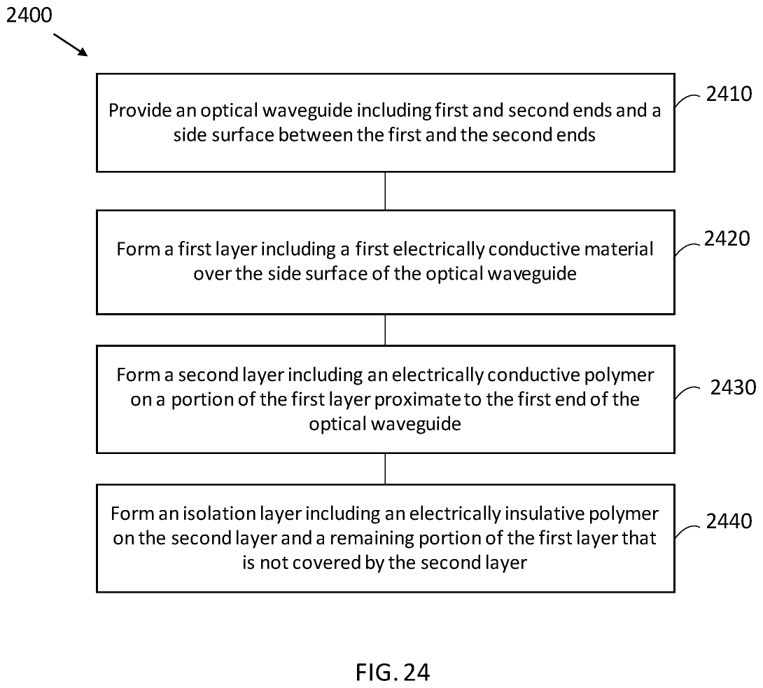

FIG. 24 shows an example method of manufacturing an electro-optical coaxial microprobe based on some embodiments of the disclosed technology.

In some implementations, a method 2400 of manufacturing an electro-optical coaxial microprobe includes, at 2410, providing an optical waveguide (e.g., an optical fiber) including first and second ends and a side surface between the first and the second ends, at 2420, forming a first layer including a first electrically conductive material over the side surface of the optical fiber, at 2430, forming a second layer including an electrically conductive polymer on a portion of the first layer proximate to the first end of the optical waveguide, and at 2440, forming an isolation layer including an electrically insulative polymer on the second layer and a remaining portion of the first layer that is not covered by the second layer.

In some implementations, the method 2400 further includes optically coupling a single-mode fiber to the second end of the optical waveguide.

In some implementations, the method 2400 further includes forming an adhesion layer including a second electrically conductive material disposed over the side surface of the optical waveguide before forming the first layer.

Therefore, various implementations of features of the disclosed technology can be made based on the above disclosure, including the examples listed below.

Example 1. An electro-optical microprobe, comprising: an optical waveguide including first and second ends and a side surface between the first and the second ends; a first layer including a first electrically conductive material disposed over the side surface of the optical waveguide; a second layer including an electrically conductive polymer disposed on a portion of the first layer proximate to the first end of the optical waveguide; and an isolation layer including an electrically insulative material disposed the second layer and a remaining portion of the first layer that is not covered by the second layer.

Example 2. The microprobe of example 1, further comprising a single-mode fiber optically coupled to the second end of the optical waveguide.

Example 3. The microprobe of example 1, wherein the optical waveguide includes a silica (SiOx) microfiber.

Example 4. The microprobe of example 1, wherein the optical waveguide includes a tin dioxide (SnO2) nanofiber.

Example 5. The microprobe of example 1, further comprising an adhesion layer including a second electrically conductive material disposed over the side surface of the optical waveguide and below the first layer.

Example 6. The microprobe of example 5, wherein the second electrically conductive material includes titanium.

Example 7. The microprobe of example 5, wherein a thickness of the adhesion layer of the first electrically conductive material is less than 100 nm.

Example 8. The microprobe of example 1, wherein the first electrically conductive material includes iridium oxide (IrOx).

Example 9. The microprobe of example 1, wherein the electrically conductive polymer includes poly(3,4-ethylene dioxythiophene)-poly(styrene sulfonate) (PEDOT:PSS) layer.

Example 10. The microprobe of example 1, wherein the electrically insulative material includes parylene.

Example 11. The microprobe of example 1, wherein a diameter of the optical waveguide is less than 100 μm.

Example 12. The microprobe of example 11, wherein the diameter of the optical waveguide is 8 μm.

Example 13. The microprobe of example 1, wherein a length of the microprobe is less than 100 mm.

Example 14. The microprobe of example 1, wherein a diameter of the microprobe at any point of the microprobe between the first end and the second end is less than 100 μm.

Example 15. The microprobe of example 1, wherein a thickness of the first layer of the first electrically conductive material is less than 1000 nm.

Example 16. The microprobe of example 1, wherein a thickness of the layer of the electrically conductive polymer is less than 1000 nm.

Example 17. The microprobe of example 1, wherein a thickness of the layer of an electrically insulative polymer is less than 10 μm.

Example 18. The microprobe of example 1, wherein the microprobe is mechanically flexible and is capable of interfacing neural networks to enable electrical and optical interrogation of the neural networks.

Example 19. The microprobe of example 18, wherein the microprobe is configured to conduct electrical measurements and provide optogenetic stimulation.

Example 20. A method of manufacturing an electro-optical coaxial microprobe, comprising: providing an optical waveguide including first and second ends and a side surface between the first and the second ends; forming a first layer including a first electrically conductive material over the side surface of the optical fiber; forming a second layer including an electrically conductive polymer on a portion of the first layer proximate to the first end of the optical waveguide; and forming an isolation layer including an electrically insulative polymer on the second layer and a remaining portion of the first layer that is not covered by the second layer.

Example 21. The method of example 20, further comprising optically coupling a single-mode fiber to the second end of the optical waveguide.

Example 22. The method of example 20, wherein the optical waveguide includes a silica (SiOx) microfiber.

Example 23. The method of example 20, wherein the optical waveguide includes a tin dioxide (SnO2) nanofiber.

Example 24. The method of example 20, further comprising forming an adhesion layer including a second electrically conductive material disposed over the side surface of the optical waveguide before forming the first layer.

Example 25. The method of example 24, wherein the second electrically conductive material includes titanium.

Example 26. The method of example 24, wherein a thickness of the adhesion layer of the first electrically conductive material is less than 100 nm.

Example 27. The method of example 20, wherein the first electrically conductive material includes iridium oxide (IrOx).

Example 28. The method of example 20, wherein the electrically conductive polymer includes poly(3,4-ethylene dioxythiophene)-poly(styrene sulfonate) (PEDOT:PSS) layer.

Example 29. The method of example 20, wherein the electrically insulative material includes parylene.

Example 30. The method of example 20, wherein a diameter of the optical waveguide is less than 100 μm.

Example 31. The method of example 30, wherein the diameter of the optical waveguide is 8 μm.

Example 32. The method of example 20, wherein a length of the microprobe is less than 100 mm.

Example 33. The method of example 20, wherein a diameter of the microprobe at any point of the microprobe between the first end and the second end is less than 100 μm.

Example 34. The method of example 20, wherein a thickness of the first layer of the first electrically conductive material is less than 1000 nm.

Example 35. The method of example 20, wherein a thickness of the layer of the electrically conductive polymer is less than 1000 nm.

Example 36. The method of example 20, wherein a thickness of the layer of an electrically insulative polymer is less than 10 μm.

Example 37. The method of example 20, wherein the microprobe is mechanically flexible and is capable of interfacing neural networks to enable electrical and optical interrogation of the neural networks.

Example 38. The method of example 37, wherein the microprobe is configured to conduct electrical measurements and provide optogenetic stimulation.

Example 39. A method of using the electro-optical coaxial microprobe of any of examples 1-19, comprising interfacing the microprobe with one or more neural networks, providing optogenetic stimulation, and conducting electrical measurements associated with a neural activity of the one or more neural networks, wherein the electro-optical coaxial microprobe has a sufficiently small length and diameter to produce negligible inflammatory response.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

It is understood that the various disclosed embodiments may be implemented individually, or collectively, in devices comprised of various optical components, electronics hardware and/or software modules and components. These devices, for example, may comprise a processor, a memory unit, an interface that are communicatively connected to each other, and may range from desktop and/or laptop computers, to mobile devices and the like. The processor and/or controller can perform various disclosed operations based on execution of program code that is stored on a storage medium. The processor and/or controller can, for example, be in communication with at least one memory and with at least one communication unit that enables the exchange of data and information, directly or indirectly, through the communication link with other entities, devices and networks. The communication unit may provide wired and/or wireless communication capabilities in accordance with one or more communication protocols, and therefore it may comprise the proper transmitter/receiver antennas, circuitry and ports, as well as the encoding/decoding capabilities that may be necessary for proper transmission and/or reception of data and other information. For example, the processor may be configured to receive electrical signals or information from the disclosed sensors (e.g., CMOS sensors), and to process the received information to produce images or other information of interest.

Various information and data processing operations described herein may be implemented in one embodiment by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Therefore, the computer-readable media that is described in the present application comprises non-transitory storage media. Generally, program modules may include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

Only a few implementations and examples are described and other implementations, enhancements and variations can be made based on what is described and illustrated in this patent document.

1. An electro-optical microprobe, comprising:
an optical waveguide including first and second ends and a side surface between the first and the second ends;
a first layer including a first electrically conductive material disposed over the side surface of the optical waveguide;
a second layer including an electrically conductive polymer disposed on a portion of the first layer proximate to the first end of the optical waveguide; and
an isolation layer including an electrically insulative material disposed the second layer and a remaining portion of the first layer that is not covered by the second layer.

2. The microprobe of claim 1, further comprising a single-mode fiber optically coupled to the second end of the optical waveguide.

3. The microprobe of claim 1, wherein the optical waveguide includes a silica (SiOₓ) microfiber or a tin dioxide (SnO₂) nanofiber.

4. (canceled)

5. The microprobe of claim 1, further comprising an adhesion layer including a second electrically conductive material disposed over the side surface of the optical waveguide and below the first layer.

6. The microprobe of claim 5, wherein the second electrically conductive material includes titanium.

7. (canceled)

8. The microprobe of claim 1, wherein the first electrically conductive material includes iridium oxide (IrOₓ).

9. The microprobe of claim 1, wherein the electrically conductive polymer includes poly(3,4-ethylene dioxythiophene)-poly(styrene sulfonate) (PEDOT:PSS) layer.

10. The microprobe of claim 1, wherein the electrically insulative material includes parylene.

11-17. (canceled)

18. The microprobe of claim 1, wherein the microprobe is mechanically flexible and is capable of interfacing neural networks to enable electrical and optical interrogation of the neural networks.

19. The microprobe of claim 18, wherein the microprobe is configured to conduct electrical measurements and provide optogenetic stimulation.

20. A method of manufacturing an electro-optical coaxial microprobe, comprising:
providing an optical waveguide including first and second ends and a side surface between the first and the second ends;
forming a first layer including a first electrically conductive material over the side surface of the optical waveguide;
forming a second layer including an electrically conductive polymer on a portion of the first layer proximate to the first end of the optical waveguide; and
forming an isolation layer including an electrically insulative polymer on the second layer and a remaining portion of the first layer that is not covered by the second layer.

21. The method of claim 20, further comprising optically coupling a single-mode fiber to the second end of the optical waveguide.

22. The method of claim 20, wherein the optical waveguide includes a silica (SiOₓ) microfiber or a tin dioxide (SnO₂) nanofiber.

23. (canceled)

24. The method of claim 20, further comprising forming an adhesion layer including a second electrically conductive material disposed over the side surface of the optical waveguide before forming the first layer.

25. The method of claim 24, wherein the second electrically conductive material includes titanium.

26. (canceled)

27. The method of claim 20, wherein the first electrically conductive material includes iridium oxide (IrOx).

28. The method of claim 20, wherein the electrically conductive polymer includes poly(3,4-ethylene dioxythiophene)-poly(styrene sulfonate) (PEDOT:PSS) layer.

29. The method of claim 20, wherein the electrically insulative material includes parylene.

30-36. (canceled)

37. The method of claim 20, wherein the microprobe is mechanically flexible and is capable of interfacing neural networks to enable electrical and optical interrogation of the neural networks.

38. The method of claim 37, wherein the microprobe is configured to conduct electrical measurements and provide optogenetic stimulation.

39. (canceled)
